(19) United States
(12) Patent Application Publication
(10) Pub. No.: US 2025/0201367 A1
(43) Pub. Date: Jun. 19, 2025

(54) NATURAL LANGUAGE CARDIOLOGY REPORTING VIA RETRIEVAL-AUGMENTED GENERATIVE ARTIFICIAL INTELLIGENCE

(71) Applicant: GE Precision Healthcare LLC, Waukesha, WI (US)

(72) Inventors: Mohit Dwivedi, Deerfield, IL (US); Vijay Kumar Reddy Arlagadda, Hoffman Estates, IL (US)

(21) Appl. No.: 18/543,562

(22) Filed: Dec. 18, 2023

(51) Int. Cl. G16H 15/00; G06F 40/40; G16H 50/20; G16H 70/20

(52) U.S. Cl. CPC G16H 15/00; G06F 40/40; G16H 50/20; G16H 70/20

(57) ABSTRACT

Systems or techniques that facilitate natural language cardiology reporting via retrieval-augmented generative artificial intelligence are provided. In various embodiments, a system can access cardiac data associated with a medical patient. In various aspects, the system can search a dynamic cardiology publication repository for one or more cardiology publications that are relevant to the cardiac data of the medical patient and that are published within a threshold margin of a current time or date. In various instances, the system can synthesize a natural language cardiology report for the medical patient, by executing a deep learning neural network on both the cardiac data and the one or more cardiology publications.

### TECHNICAL FIELD

The subject disclosure relates generally to cardiology reporting, and more specifically to natural language cardiology reporting via retrieval-augmented generative artificial intelligence.

### BACKGROUND

When given a medical patient that presents with cardiovascular-related symptoms, a cardiology report for the medical patient can be warranted. Existing techniques generate such a cardiology report either manually or via automated template-filling. Because the field of cardiology is a burgeoning, constantly evolving area of medicine, the cardiology reports of such existing techniques often do not account for state-of-the-art findings in the field of cardiology.

Accordingly, systems or techniques that can address one or more of these technical problems can be desirable.

### SUMMARY

The following presents a summary to provide a basic understanding of one or more embodiments. This summary is not intended to identify key or critical elements, or delineate any scope of the particular embodiments or any scope of the claims. Its sole purpose is to present concepts in a simplified form as a prelude to the more detailed description that is presented later. In one or more embodiments described herein, devices, systems, computer-implemented methods, apparatus or computer program products that facilitate systems and methods for providing natural language cardiology reporting via retrieval-augmented generative artificial intelligence are described.

According to one or more embodiments, a system is provided. The system can comprise a non-transitory computer-readable memory that can store computer-executable components. The system can further comprise a processor that can be operably coupled to the non-transitory computer-readable memory and that can execute the computer-executable components stored in the non-transitory computer-readable memory. In various embodiments, the computer-executable components can comprise an access component that can access cardiac data associated with a medical patient. In various aspects, the computer-executable components can comprise a retrieval component that can search a dynamic cardiology publication repository for one or more cardiology publications that are relevant to the cardiac data of the medical patient and that are published within a threshold margin of a current time or date. In various instances, the computer-executable components can comprise a generative component that can synthesize a natural language cardiology report for the medical patient, by executing a deep learning neural network on both the cardiac data and the one or more cardiology publications.

According to one or more embodiments, a computer-implemented method is provided. In various embodiments, the computer-implemented method can comprise accessing, by a device operatively coupled to a processor, cardiac data associated with a medical patient. In various aspects, the computer-implemented method can comprise searching, by the device, a dynamic cardiology publication repository for one or more cardiology publications that are relevant to the cardiac data of the medical patient and that are published within a threshold margin of a current time or date. In various instances, the computer-implemented method can comprise synthesizing, by the device and by executing a deep learning neural network on both the cardiac data and the one or more cardiology publications, a natural language cardiology report for the medical patient.

According to one or more embodiments, a computer program product for facilitating natural language medical reporting via retrieval-augmented generative artificial intelligence is provided. In various embodiments, the computer program product can comprise a non-transitory computer-readable memory having program instructions embodied therewith. In various aspects, the program instructions can be executable by a processor to cause the processor to access medical data associated with a medical patient. In various instances, the program instructions can be further executable to cause the processor to search a dynamic medical publication repository for one or more medical publications that are relevant to the medical data of the medical patient and that are published within a threshold margin of a current time or date. In various cases, the program instructions can be further executable to cause the processor to synthesize a natural language medical report for the medical patient, by executing a deep learning neural network on both the medical data and the one or more medical publications.

### DESCRIPTION OF THE DRAWINGS

FIG. 1 illustrates a block diagram of an example, non-limiting system that facilitates natural language cardiology reporting via retrieval-augmented generative artificial intelligence in accordance with one or more embodiments described herein.

FIG. 2 illustrates a block diagram of an example, non-limiting system including a dynamic cardiology publication repository and one or more relevant cardiology publications that facilitates natural language cardiology reporting via retrieval-augmented generative artificial intelligence in accordance with one or more embodiments described herein.

FIG. 3 illustrates an example, non-limiting block diagram showing how one or more relevant cardiology publications can be identified in a dynamic cardiology publication repository in accordance with one or more embodiments described herein.

FIG. 4 illustrates a block diagram of an example, non-limiting system including a deep learning neural network and a natural language cardiology report that facilitates natural language cardiology reporting via retrieval-augmented generative artificial intelligence in accordance with one or more embodiments described herein.

FIG. 5 illustrates an example, non-limiting block diagram showing how a natural language cardiology report can be generated in accordance with one or more embodiments described herein.

FIG. 6 illustrates a block diagram of an example, non-limiting system including a training component and a training dataset that facilitates natural language cardiology reporting via retrieval-augmented generative artificial intelligence in accordance with one or more embodiments described herein.

FIG. 7 illustrates an example, non-limiting block diagram of a training dataset in accordance with one or more embodiments described herein.

FIG. 8 illustrates an example, non-limiting block diagram showing how a deep learning neural neural network can be trained in accordance with one or more embodiments described herein.

FIG. 9 illustrates a flow diagram of an example, non-limiting computer-implemented method that facilitates natural language cardiology reporting via retrieval-augmented generative artificial intelligence in accordance with one or more embodiments described herein.

FIG. 10 illustrates a block diagram of an example, non-limiting operating environment in which one or more embodiments described herein can be facilitated.

FIG. 11 illustrates an example networking environment operable to execute various implementations described herein.

### DETAILED DESCRIPTION

The following detailed description is merely illustrative and is not intended to limit embodiments or application/uses of embodiments. Furthermore, there is no intention to be bound by any expressed or implied information presented in the preceding Background or Summary sections, or in the Detailed Description section.

One or more embodiments are now described with reference to the drawings, wherein like referenced numerals are used to refer to like elements throughout. In the following description, for purposes of explanation, numerous specific details are set forth in order to provide a more thorough understanding of the one or more embodiments. It is evident, however, in various cases, that the one or more embodiments can be practiced without these specific details.

A medical patient can present with cardiovascular-related symptoms (e.g., chest pain, shortness of breath, exercise intolerance). In such cases, a cardiology report for the medical patient can be warranted. In various aspects, cardiovascular-related data of the medical patient can be measured (e.g., heart rate, blood pressure), and a cardiology report can be prepared based on such measured data, where the cardiology report can be a natural language, plain text, or otherwise human-readable document that summarizes the cardiovascular health of the medical patient or that otherwise conveys any cardiac-related diagnoses or prognoses for the medical patient.

Some existing techniques generate such a cardiology report manually. That is, a medical professional (e.g., physician, nurse) manually writes or types the cardiology report using word processing software of a computer.

Other existing techniques generate such a cardiology report via automated template-filling. That is, a cardiology report template having fixed, pre-defined text fields or check-boxes can be created by a medical professional, and such fixed, pre-defined text fields or check-boxes can be automatically filled based on the measured cardiovascular-related data of the medical patient.

In any case, the field of cardiology is a burgeoning, constantly evolving area of medicine. Indeed, cutting-edge cardiology research is often released on a monthly, weekly, or even daily basis. Accordingly, the cardiology reports of existing techniques (whether manual or template-based) often fail to account for state-of-the-art findings in the field of cardiology.

Accordingly, systems or techniques that can address one or more of these technical problems can be desirable.

Various embodiments described herein can address one or more of these technical problems. One or more embodiments described herein can include systems, computer-implemented methods, apparatus, or computer program products that can facilitate natural language cardiology reporting via retrieval-augmented generative artificial intelligence. In particular, various embodiments described herein can involve configuring or training a generative machine learning model to synthesize cardiology reports based on inputted patient cardiac data. However, rather than being alone, the inputted patient cardiac data can be accompanied by one or more recently-released cardiology publications (e.g., recently-published cardiology research papers, recently-published cardiology journal articles) that are relevant to the inputted patient cardiac data. Accordingly, any cardiology report synthesized by the generative machine learning model can be considered as reflecting not just the specific cardiovascular data that is measured for any given medical patient, and not just whatever specific cardiology diagnostic or prognostic insights that the generative machine learning model encountered during training. Instead, any cardiology report synthesized by the generative machine learning model can be considered as also reflecting any newly-published cardiology diagnostic or prognostic insights, even though the generative machine learning model did not encounter such new insights during training. In other words, the generative machine learning model can be considered as synthesizing cardiology reports for medical patients, by leveraging cutting-edge cardiology research findings that might have pertinent applications to those medical patients. In this way, clinically-up-to-date or state-of-the-art cardiology reports can be automatically generated for any given medical patient, unlike with existing techniques.

Various embodiments described herein can be considered as a computerized tool (e.g., any suitable combination of computer-executable hardware or computer-executable software) that can facilitate natural language cardiology reporting via retrieval-augmented generative artificial intelligence. In various aspects, such computerized tool can comprise an access component, a retrieval component, a generative component, or a result component.

In various embodiments, there can be cardiac data. In various aspects, the cardiac data can correspond to any suitable medical patient. In various instances, the cardiac data can indicate, quantify, convey, or otherwise represent any suitable measurable medical information regarding a cardio-vasculature of the medical patient. In some cases, the cardiac data can comprise any suitable metadata of the medical patient (e.g., age, sex, weight, height, health habits, pathologies, prostheses, symptoms). In some aspects, the cardiac data can comprise any suitable cardiac signal data of the medical patient (e.g., heart rate, electrocardiogram). In some instances, the cardiac data can comprise any suitable cardiac image data of the medical patient (e.g., computed tomography (CT) cardiac images, echocardiogram images). In various cases, any of the cardiac data can be measured or otherwise captured by any suitable medical diagnostic equipment (e.g., pulse monitors, electrocardiogram monitors, CT scanners, echocardiogram scanners).

In various aspects, it can be desired to automatically synthesize a natural language cardiology report for the medical patient, based on the cardiac data. In various cases, the computerized tool can facilitate such synthesis as described herein.

In various embodiments, the access component of the computerized tool can electronically receive or otherwise electronically access the cardiac data. In some aspects, the access component can electronically retrieve the cardiac data from any suitable centralized or decentralized data structures (e.g., graph data structures, relational data structures, hybrid data structures), whether remote from or local to the access component. In other aspects, the access component can electronically retrieve the cardiac data from whatever medical diagnostic equipment measures or captures the cardiac data. In any case, the access component can electronically obtain or access the cardiac data, such that the access component can serve as a conduit by which or through which other components of the computerized tool can electronically interact with (e.g., read, write, edit, copy, manipulate) the cardiac data.

In various embodiments, the retrieval component of the computerized tool can electronically search through a dynamic cardiology publication repository, so as to identify one or more cardiology publications that are recent and relevant to the cardiac data.

In various aspects, a cardiology publication can be any suitable natural language or plain text electronic document, or portion thereof, that can describe, convey, or otherwise contain any suitable medical or clinical research, studies, or experimental results that are performed or conducted by any suitable entities in the field of cardiology. As a non-limiting example, the dynamic cardiology publication repository can be or otherwise correspond to a scientific cardiology website (e.g., cardiology website of the World Health Organization, website of the American Journal of Cardiology), and a cardiology publication can be any suitable research paper, journal article, or portion thereof that is published by that scientific cardiology website.

In various cases, the dynamic cardiology publication repository can be routinely, regularly, continually, or continuously updated with new cardiology publications, hence the term “dynamic”. As some non-limiting examples, new cardiology publications (e.g., new cardiology research papers, new cardiology journal articles) can be added to or published by the dynamic cardiology publication repository on a monthly basis, on a weekly basis, on a daily basis, or even on an hourly basis. In other words, the dynamic cardiology publication repository can be considered as not being a static dataset.

In various aspects, the retrieval component can filter through the dynamic cardiology publication repository, so as to distinguish between cardiology publications that are recent and those that are not recent. In various instances, the retrieval component can facilitate such filtering based on a threshold temporal margin (e.g., 24-hour margin, 1-week margin) that defines whether or not a cardiology publication qualifies as recent. If a cardiology publication was published by or added to the dynamic cardiology publication repository within that threshold temporal margin of a current time or date, then such cardiology publication can be considered as recent. In contrast, if the cardiology publication was instead published by or added to the dynamic cardiology publication repository outside of that threshold temporal margin of the current time or date, then such cardiology publication can be considered as not recent. As a non-limiting example, the threshold temporal margin can be 24 hours. In such case, any cardiology publication that was published by or added to the dynamic cardiology publication repository less than 24 hours ago can be considered as recent, whereas any cardiology publication that was published by or added to the dynamic cardiology publication repository more than 24 hours ago can be considered as not recent. As another non-limiting example, the threshold temporal margin can be one week. In such case, any cardiology publication that was published by or added to the dynamic cardiology publication repository less than one week ago can be considered as recent, whereas any cardiology publication that was published by or added to the dynamic cardiology publication repository more than one week ago can be considered as not recent.

In various cases, after filtering the dynamic cardiology publication repository for recency, the retrieval component can search through the dynamic cardiology publication repository, so as to identify one or more recent cardiology publications that are substantively relevant, related, or applicable to the cardiac data. In various aspects, the retrieval component can facilitate such searching via any suitable document search techniques (e.g., keyword-based document search techniques, embedding-based document search techniques, probabilistic retrieval-based document search techniques). In various instances, a cardiology publication can be considered as being relevant, related, or applicable to the cardiac data if the textual content of that cardiology publication semantically pertains in some way to the metadata, to the cardiac signal data, or to the cardiac image data of the medical patient.

As a non-limiting example, suppose that the metadata indicates that the medical patient is an elderly male that habitually smokes tobacco. In such case, a cardiology publication that discusses new clinical research regarding geriatric smokers can be considered as being relevant, related, or applicable to the cardiac data of the medical patient.

As another non-limiting example, suppose that the metadata indicates that the medical patient is a weightlifter outfitted with a pacemaker. In such case, a cardiology publication that discusses new physical exercise restrictions for people with pacemakers can be considered as being relevant, related, or applicable to the cardiac data of the medical patient.

As even another non-limiting example, suppose that the cardiac data includes a heart rate of the medical patient and an electrocardiogram trace of the medical patient. In such case, a cardiology publication that discusses a new cardiology metric that is a function of both heart rate and electrocardiogram trace can be considered as being relevant, related, or applicable to the cardiac data of the medical patient.

As still another non-limiting example, suppose that the cardiac data includes an echocardiogram image of the medical patient. In such case, a cardiology publication that discusses a new cardiology prognostic insight that can be derived from echocardiogram pixels or voxels can be considered as being relevant, related, or applicable to the cardiac data of the medical patient.

In any case, the retrieval component can identify in the dynamic cardiology publication repository one or more recent and relevant cardiology publications, based on the cardiac data of the medical patient.

In various embodiments, the generative component of the computerized tool can electronically store, maintain, control, or otherwise access a deep learning neural network. In various aspects, the deep learning neural network can exhibit any suitable deep learning internal architecture. For example, the deep learning neural network can include any suitable numbers of any suitable types of layers (e.g., input layer, one or more hidden layers, output layer, any of which can be convolutional layers, dense layers, non-linearity layers, long short-term memory (LSTM) layers, pooling layers, batch normalization layers, or padding layers). As another example, the deep learning neural network can include any suitable numbers of neurons in various layers (e.g., different layers can have the same or different numbers of neurons as each other). As yet another example, the deep learning neural network can include any suitable activation functions (e.g., softmax, sigmoid, hyperbolic tangent, rectified linear unit) in various neurons (e.g., different neurons can have the same or different activation functions as each other). As still another example, the deep learning neural network can include any suitable interneuron connections or interlayer connections (e.g., forward connections, skip connections, recurrent connections).

In various aspects, the deep learning neural network can be configured to synthesize cardiology reports, based on inputted cardiac data, and also based on any suitable electronic documents that accompany such inputted cardiac data. Accordingly, the generative component can electronically execute the deep learning neural network on both the cardiac data of the medical patient and the one or more recent and relevant cardiology publications. In various instances, such execution can cause the deep learning neural network to produce as output a natural language cardiology report for the medical patient. More specifically, the generative component can concatenate the cardiac data and the one or more recent and relevant cardiology publications together and can feed such concatenation to an input layer of the deep learning neural network. In various cases, such concatenation can complete a forward pass through one or more hidden layers of the deep learning neural network. In various aspects, an output layer of the deep learning neural network can calculate the natural language cardiology report, based on activation maps provided by the one or more hidden layers.

In various instances, the natural language cardiology report can be an electronic document that is written or formatted in human-readable or plain text prose, where such human-readable or plain text prose summarizes the cardiovascular health of the medical patient. In particular, the deep learning neural network can make any suitable diagnostic or prognostic inferences based on the cardiac data of the medical patient, and the natural language cardiology report can textually describe or explain the cardiac data and those diagnostic or prognostic inferences. In various cases, the natural language cardiology report can exhibit any suitable organization or formatting (e.g., patient characteristics section, paragraph, or bullet points; cardiac measurements section, paragraph, or bullet points; cardiac images section, paragraph, or bullet points; findings or diagnosis section, paragraph, or bullet points; recommended treatment section, paragraph, or bullet points; impressions or prognosis section, paragraph, or bullet points).

In various aspects, the natural language cardiology report can include not just information gleaned or inferred from the cardiac data of the medical patient, but also information gleaned or inferred from the one or more recent and relevant cardiology publications. Because the one or more recent and relevant cardiology publications can convey or indicate cutting-edge research findings in the field of cardiology, the natural language cardiology report can be considered as being clinically up-to-date or otherwise as reflecting or being consistent with the most recent cardiological research. Note that this can be the case, notwithstanding that the deep learning neural network might not have yet been trained on such most recent cardiological research.

As a non-limiting example, suppose that the cardiac data indicates that the medical patient has a pacemaker and has an electrocardiogram trace that exceeds some threshold. Furthermore, suppose that, at the time that the deep learning neural network was trained, it was believed in the field of cardiology that those with pacemakers and such electrocardiogram traces should perform no more than 30 minutes of strenuous exercise per day. Further still, suppose that the one or more recent and relevant cardiology publications includes a peer-reviewed study indicating that, contrary to previous cardiology teachings, those with pacemakers and such electrocardiogram traces can safely perform up to 1.5 hours of strenuous exercise per day. In such case, execution of the deep learning neural network on both the cardiac data and the one or more recent and relevant cardiology publications can cause the natural language cardiology report to describe the 1.5-hour exercise restriction rather than the 30-minute exercise restriction. In other words, the deep learning neural network can be considered as applying to the cardiac data of the medical patient whatever cutting-edge, up-to-date clinical information is indicated by the one or more recent and relevant cardiology publications, notwithstanding that the deep learning neural network did not encounter such cutting-edge, up-to-date clinical information during training.

As another non-limiting example, suppose that the cardiac data indicates that the medical patient is a newborn with a cardiac lesion measuring more than a threshold diameter. Furthermore, suppose that, at the time that the deep learning neural network was trained, it was believed in the field of cardiology that newborns with such cardiac lesions should undergo open-heart surgery. Further still, suppose that the one or more recent and relevant cardiology publications includes a peer-reviewed article indicating that, contrary to previous cardiology teachings, newborns with such cardiac lesions can be successfully treated using a new minimally-invasive surgical procedure. In such case, execution of the deep learning neural network on both the cardiac data and the one or more recent and relevant cardiology publications can cause the natural language cardiology report to recommend that the medical patient undergo the new minimally-invasive surgical procedure rather than open-heart surgery. Again, the deep learning neural network can be considered as applying to the cardiac data of the medical patient whatever cutting-edge, up-to-date clinical information is indicated by the one or more recent and relevant cardiology publications, notwithstanding that the deep learning neural network did not encounter such cutting-edge, up-to-date clinical information during training.

In any case, the generative component can execute the deep learning neural network on both the cardiac data and the one or more recent and relevant cardiology publications, thereby synthesizing the natural language cardiology report.

In various embodiments, the result component of the computerized tool can electronically facilitate any suitable actions, based on the natural language cardiology report. In some cases, the result component can visually render the natural language cardiology report on any suitable electronic display. In other cases, the result component can electronically transmit the natural language cardiology report to any suitable computing device.

To help cause the natural language cardiology report to be accurate, the deep learning neural network can first undergo training. In various aspects, the computerized tool described herein can facilitate such training in any suitable fashion (e.g., in supervised fashion) based on any suitable training dataset.

Various embodiments described herein can be employed to use hardware or software to solve problems that are highly technical in nature (e.g., to facilitate natural language cardiology reporting via retrieval-augmented generative artificial intelligence), that are not abstract and that cannot be performed as a set of mental acts by a human. Further, some of the processes performed can be performed by a specialized computer (e.g., document search engine that can perform keyword searches or embedding searches; deep learning neural network having internal parameters such as convolutional kernels) for carrying out defined acts related to cardiology reporting. For example, such defined acts can include: accessing, by a device operatively coupled to a processor, cardiac data associated with a medical patient; searching, by the device, a dynamic cardiology publication repository for one or more cardiology publications that are relevant to the cardiac data of the medical patient and that are published within a threshold margin of a current time or date; and synthesizing, by the device and by executing a deep learning neural network on both the cardiac data and the one or more cardiology publications, a natural language cardiology report for the medical patient.

Such defined acts are not performed manually by humans. Indeed, neither the human mind nor a human with pen and paper can: electronically search a dynamic document repository for cardiology publications (e.g., cardiology research papers, cardiology journal articles) that are recently-published and pertinent to the cardiac data of a medical patient; and electronically execute a generative machine learning model on both the cardiac data and the recent and pertinent cardiology publications, thereby yielding a plain text cardiology report for the medical patient. Indeed, a generative machine learning model (e.g., a deep learning neural network) is an inherently-computerized construct that simply cannot be meaningfully executed or trained in any way by the human mind without computers. Similarly, electronic document searching (e.g., based on keywords, embeddings, or word probabilities) is an inherently-computerized activity that deals with enabling computers to substantively search or sift through document databases. Electronic document searching cannot be meaningfully implemented in any way by the human mind without computers. Accordingly, a computerized tool that can obtain cardiac data of a patient, search for electronic cardiology research papers or journal articles that are recently-published and substantively relevant to that cardiac data, and execute a generative machine learning model on both that cardiac data and the recently-published and relevant electronic cardiology research papers or journal articles, thereby yielding a plain text cardiology report for the patient, is likewise inherently-computerized and cannot be implemented in any sensible, practical, or reasonable way without computers.

Moreover, various embodiments described herein can integrate into a practical application various teachings relating to natural language cardiology reporting via retrieval-augmented generative artificial intelligence. As described above, existing techniques generate cardiology reports either manually or via automated template-filling. Unfortunately, the cardiology reports generated by such existing techniques often do not include or reflect cutting-edge or state-of-the-art cardiology research, given how rapidly research and scientific discoveries progress in the field of cardiology.

As the present inventors recognized, such failure of existing cardiology reporting techniques to remain up-to-date on the latest advancements in the field of cardiology can be ameliorated by implementation or adaptation of retrieval-augmented generative artificial intelligence. In particular, various embodiments described herein can involve configuring a deep learning neural network to generate or synthesize plain text cardiology reports based on inputted patient cardiac data (e.g., inputted patient metadata, inputted patient heart rates, inputted patient echocardiograms). However, in various aspects, various embodiments described herein can involve utilizing any suitable document search engine (e.g., keyword engine, embedding engine, probabilistic engine) to identify or fetch one or more cardiology publications (e.g., research papers, journal articles) that are recently published and that are substantively relevant to the patient cardiac data. In various instances, such one or more identified or fetched cardiology publications can be considered as describing various state-of-the-art or cutting-edge advancements in the field of cardiology that are or might be applicable to the patient cardiac data. In various cases, various embodiments described herein can involve executing the deep learning neural network on both the patient cardiac data and the one or more identified or fetched cardiology publications. In various aspects, such execution can cause the deep learning neural network to synthesize as output a plain text cardiology report, where such plain text cardiology report can be considered as describing or summarizing a cardiovascular health indicated by the patient cardiac data in light of the one or more identified or fetched cardiology publications (e.g., in light of the latest relevant advancements in the field of cardiology). In this way, clinically up-to-date cardiology reports can be automatically generated for any given medical patient. Contrast this with existing techniques whose manual or template-based cardiology reports often do not reflect or take into account the latest advancements in the field of cardiology. Indeed, a medical professional that manually writes a cardiology report can be unaware of the most recent and relevant cardiology advancements. Furthermore, the number and types of text fields or check-boxes in a cardiology report template are fixed at particular moment in time; thus, the cardiology report template does not take into account any cardiology advancements that are discovered after creation or preparation of the cardiology report template.

For at least these reasons, various embodiments described herein facilitate improved cardiology reporting, as compared to existing techniques. Thus, various embodiments described herein certainly constitute a tangible and concrete technical improvement or technical advantage in the field of cardiology reporting. Accordingly, such embodiments clearly qualify as useful and practical applications of computers.

Furthermore, various embodiments described herein can control real-world tangible devices based on the disclosed teachings. For example, various embodiments described herein can electronically train or execute real-world deep learning neural networks on real-world cardiac data and cardiology publications, and can electronically render real-world results (e.g., plain text cardiology reports) on real-world computer screens.

It should be appreciated that the herein figures and description provide non-limiting examples of various embodiments and are not necessarily drawn to scale.

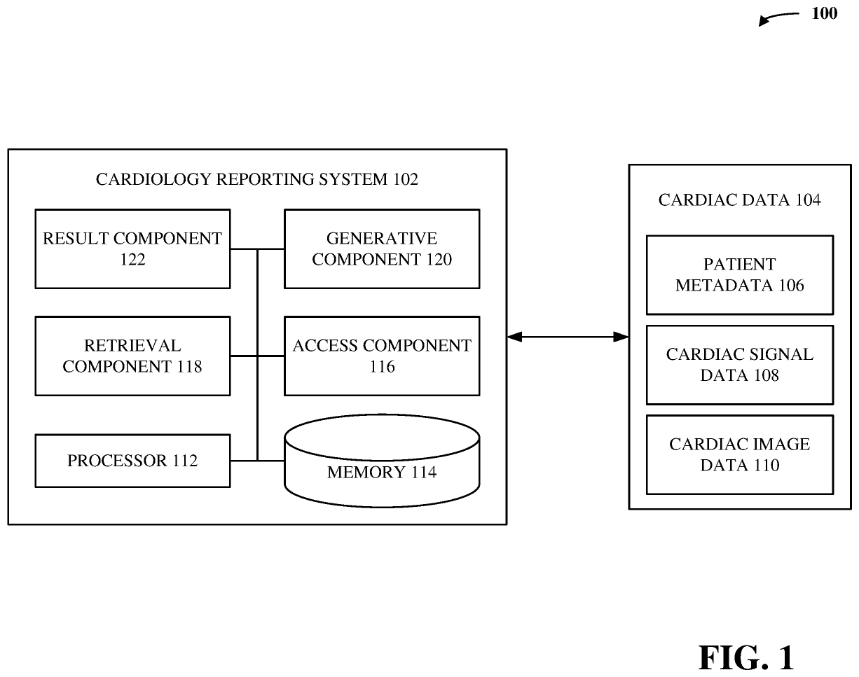

FIG. 1 illustrates a block diagram of an example, non-limiting system 100 that can facilitate natural language cardiology reporting via retrieval-augmented generative artificial intelligence in accordance with one or more embodiments described herein. As shown, a cardiology reporting system 102 can be electronically integrated, via any suitable wired or wireless electronic connections, with cardiac data 104.

In various embodiments, the cardiac data 104 can correspond to or be associated with any suitable medical patient (e.g., human, animal, or otherwise). In various aspects, the cardiac data 104 can be any suitable electronic data that indicates, conveys, or otherwise represents any suitable measurable or quantifiable biological information or attributes that are or might be related to a cardiovascular health of the medical patient.

In various instances, as shown, the cardiac data 104 can comprise patient metadata 106. In various cases, the patient metadata 106 can be one or more scalars, one or more vectors, one or more matrices, one or more tensors, one or more character strings, or any suitable combination thereof that indicates, conveys, or otherwise represents any suitable physical characteristics or attributes of the medical patient. As a non-limiting example, the patient metadata 106 can indicate, convey, or otherwise represent an age or age range of the medical patient. As another non-limiting example, the patient metadata 106 can indicate, convey, or otherwise represent a biological sex or gender of the medical patient. As even another non-limiting example, the patient metadata 106 can indicate, convey, or otherwise represent a weight or mass of the medical patient. As still another non-limiting example, the patient metadata 106 can indicate, convey, or otherwise represent a height or width of the medical patient. As yet another non-limiting example, the patient metadata 106 can indicate, convey, or otherwise represent a body fat percentage of the medical patient. As another non-limiting example, the patient metadata 106 can indicate, convey, or otherwise represent one or more health habits or lifestyle habits of the medical patient, such as: how much or what types, if any, of tobacco the medical patient uses on a daily, weekly, monthly, or yearly basis; how much or what types, if any, of physical exercise the medical patient performs on a daily, weekly, monthly, or yearly basis; or how much or what types, if any, of alcohol the medical patient consumes on a daily, weekly, monthly, or yearly basis. As still another non-limiting example, the patient metadata 106 can indicate, convey, or otherwise represent one or more health statuses of the medical patient, such as: whether or not the medical patient is pregnant; how far along in pregnancy the medical patient is; whether or not the medical patient has a known pathology (e.g., cancer, heart disease, diabetes, asthma); or how severe such known pathology is (e.g., metastasized versus non-metastasized cancer). As even another non-limiting example, the patient metadata 106 can indicate, convey, or otherwise represent one or more prostheses or implants that the medical patient possesses, such as: prosthetic limbs; a pacemaker; intravenous stents; or spinal rods. As yet another non-limiting example, the patient metadata 106 can indicate, convey, or otherwise represent one or more symptoms of which the medical patient complains, such as: shortness of breath; exercise intolerance; or chest pains. In various cases, the patient metadata 106 can be typed into or otherwise accessed by any suitable computing device having any suitable human-computer interface, such as: a desktop computer; a laptop computer; a smart phone; or a tablet device.

In various aspects, as shown, the cardiac data 104 can comprise cardiac signal data 108. In various instances, the cardiac signal data 108 can be one or more scalars, one or more vectors, one or more matrices, one or more tensors, one or more character strings, or any suitable combination thereof that indicates, conveys, or otherwise represents any suitable measurable behavior or activity that is exhibited by a heart of the medical patient. As a non-limiting example, the cardiac signal data 108 can indicate, convey, or otherwise represent a pulse rate of the medical patient (e.g., an instantaneous pulse rate of the medical patient; an average pulse rate of the medical patient; or a timeseries of multiple instantaneous or average pulse rates of the medical patient recorded over a time interval). As another non-limiting example, the cardiac signal data 108 can indicate, convey, or otherwise represent a blood pressure of the medical patient (e.g., an instantaneous blood pressure of the medical patient; an average blood pressure of the medical patient; or a timeseries of multiple instantaneous or average blood pressures of the medical patient recorded over a time interval). As still another non-limiting example, the cardiac signal data 108 can indicate, convey, or otherwise represent an electrocardiogram trace of the medical patient (e.g., a timeseries showing cardiac muscle depolarization and repolarization cycles of the medical patient recorded over a time interval). As even another non-limiting example, the cardiac signal data 108 can indicate, convey, or otherwise represent a seismocardiogram trace of the medical patient (e.g., a timeseries showing myocardial-induced chest wall acceleration and deceleration cycles of the medical patient recorded over a time interval). As yet another non-limiting example, the cardiac signal data 108 can indicate, convey, or otherwise represent a photoplethysmogram trace of the medical patient (e.g., a timeseries showing cardiac volume change cycles of the medical patient recorded over a time interval). As another non-limiting example, the cardiac signal data 108 can indicate, convey, or otherwise represent a phonocardiogram trace of the medical patient (e.g., a timeseries showing cardiac sounds or cardiac murmurs of the medical patient recorded over a time interval). In various cases, the cardiac signal data 108 can be measured or otherwise captured by any suitable medical equipment or medical hardware, such as: pulse monitors; blood pressure monitors; electrocardiogram monitors; seismocardiogram monitors; photoplethysmogram monitors; or phonocardiogram monitors.

In various aspects, as shown, the cardiac data 104 can comprise cardiac image data 110. In various instances, the cardiac image data 110 can be one or more scalars, one or more vectors, one or more matrices, one or more tensors, one or more character strings, or any suitable combination thereof that indicates, conveys, or otherwise represents any suitable medical scanned images of the heart of the medical patient. As a non-limiting example, the cardiac image data 110 can indicate, convey, or otherwise represent an echocardiogram pixel array or voxel array depicting the heart of the medical patient. As another non-limiting example, the cardiac image data 110 can indicate, convey, or otherwise represent a computed tomography (CT) pixel array or voxel array depicting the heart of the medical patient. As even another non-limiting example, the cardiac image data 110 can indicate, convey, or otherwise represent a magnetic resonance imaging (MRI) pixel array or voxel array depicting the heart of the medical patient. As still another non-limiting example, the cardiac image data 110 can indicate, convey, or otherwise represent a positron emission tomography (PET) pixel array or voxel array depicting the heart of the medical patient. As even another non-limiting example, the cardiac image data 110 can indicate, convey, or otherwise represent a nuclear medicine (NM) pixel array or voxel array depicting the heart of the medical patient. In various cases, the cardiac image data 110 can be captured or otherwise generated by any suitable medical imaging equipment or scanners, such as: ultrasound scanners; CT scanners; MRI scanners; PET scanners; or NM scanners.

In various aspects, it can be desired to generate a cardiology report for the medical patient, based on the cardiac data 104. In various instances, as described herein, the cardiology reporting system 102 can facilitate such generation.

In various embodiments, the cardiology reporting system 102 can comprise a processor 112 (e.g., computer processing unit, microprocessor) and a non-transitory computer-readable memory 114 that is operably or operatively or communicatively connected or coupled to the processor 112. The non-transitory computer-readable memory 114 can store computer-executable instructions which, upon execution by the processor 112, can cause the processor 112 or other components of the cardiology reporting system 102 (e.g., access component 116, retrieval component 118, generative component 120, result component 122) to perform one or more acts. In various embodiments, the non-transitory computer-readable memory 114 can store computer-executable components (e.g., access component 116, retrieval component 118, generative component 120, result component 122), and the processor 112 can execute the computer-executable components.

In various embodiments, the cardiology reporting system 102 can comprise an access component 116. In various aspects, the access component 116 can electronically receive or otherwise electronically access the cardiac data 104. In various instances, the access component 116 can electronically retrieve the cardiac data 104 from any suitable centralized or decentralized data structures (not shown) or from any suitable centralized or decentralized computing devices (not shown), whether local to or remote from the access component 116. As a non-limiting example, the access component 116 can electronically retrieve the cardiac data 104 from whatever computing devices (e.g., desktop computer, laptop computer, smart phone, tablet) or medical equipment (e.g., pulse monitors, electrocardiogram monitors, CT scanners, ultrasound scanners) that were used to measure, capture, or create the cardiac data 104. In any case, the access component 116 can electronically obtain or access the cardiac data 104, such that other components of the cardiology reporting system 102 can electronically interact (e.g., by proxy) with the cardiac data 104.

In various embodiments, the cardiology reporting system 102 can comprise a retrieval component 118. In various aspects, the retrieval component 118 can, as described herein, identify one or more recent cardiology publications that are relevant to the cardiac data 104.

In various embodiments, the cardiology reporting system 102 can comprise a generative component 120. In various instances, the generative component 120 can, as described herein, execute a deep learning neural network on both the cardiac data 104 and the one or more recent and relevant cardiology publications, thereby yielding a natural language cardiology report for the medical patient.

In various embodiments, the cardiology reporting system 102 can comprise a result component 122. In various cases, the result component 122 can, as described herein, initiate any suitable electronic actions based on the natural language cardiology report.

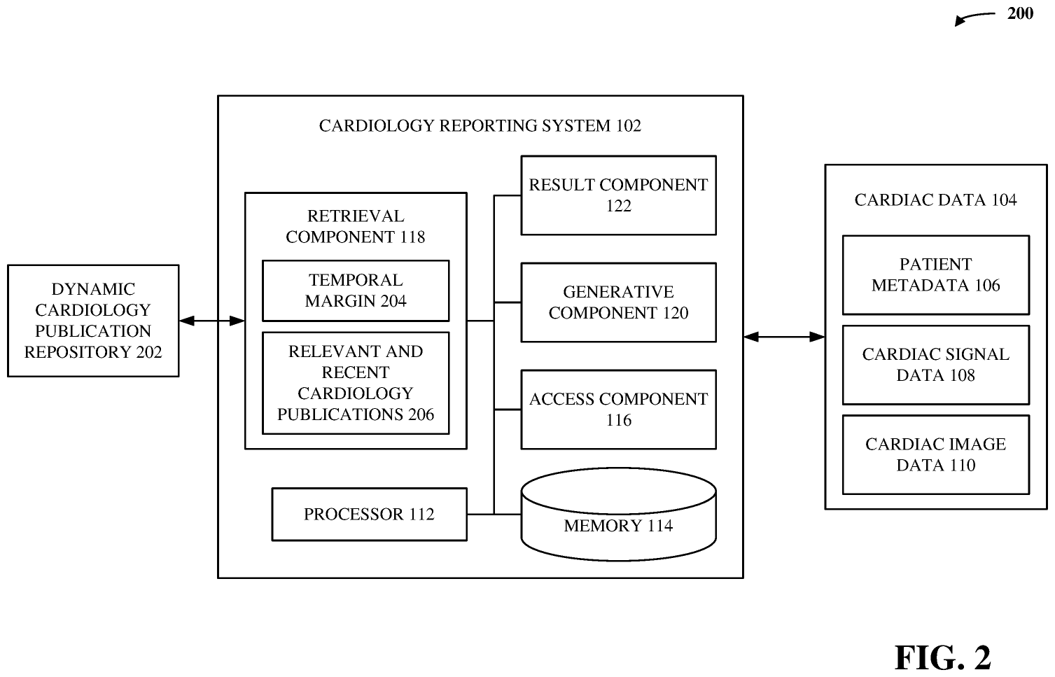

FIG. 2 illustrates a block diagram of an example, non-limiting system 200 including a dynamic cardiology publication repository and one or more relevant cardiology publications that can facilitate natural language cardiology reporting via retrieval-augmented generative artificial intelligence in accordance with one or more embodiments described herein. As shown, the system 200 can, in some cases, comprise the same components as the system 100, and can further comprise a dynamic cardiology publication repository 202, a temporal margin 204, or one or more relevant and recent cardiology publications 206.

In various embodiments, the retrieval component 118 can electronically communicate with the dynamic cardiology publication repository 202. In various aspects, the dynamic cardiology publication repository 202 can be any suitable electronic database or electronic data structure that can electronically store, electronically maintain, electronically host, electronically publish, electronically share, or otherwise electronically disseminate cardiology publications. As a non-limiting example, the dynamic cardiology publication repository 202 can be an internet-accessible website of a medical organization, such as: a website of the World Health Organization; a website of the Centers for Disease Control and Prevention; a website of the American Medical Association; or a website of the American Journal of Cardiology. In any case, a cardiology publication can be any suitable electronic document, or portion thereof, that is written in plain text, natural language, or prose, and that textually describes or explains any suitable academic or clinical information that is pertinent to the field of cardiology. As a non-limiting example, a cardiology publication can be a peer-reviewed research paper or a peer-reviewed journal article that describes or explains scientific, experimental, numerical, or theoretical findings or discoveries in the field of cardiology.

In various instances, new cardiology publications can be added to the dynamic cardiology publication repository 202 in regular, periodic, continuous, or continual fashion. In other instances, new cardiology publications can instead be added to the dynamic cardiology publication repository 202 in irregular, aperiodic, or otherwise ad hoc fashion. In any case, the content of the dynamic cardiology publication repository 202 can increase, or otherwise be updated over time, rather than staying statically frozen in time. Accordingly, the dynamic cardiology publication repository 202 can be considered as containing or representing cutting-edge or up-to-date scientific discoveries or research findings in the field of cardiology.

In various aspects, the retrieval component 118 can electronically search through the dynamic cardiology publication repository 202, so as to identify or locate one or more cardiology publications that are both recently published and substantively relevant to the cardiac data 104. In various cases, such identified or located publications can be referred to as the one or more relevant and recent cardiology publications 206.

More specifically, the retrieval component 118 can, in various instances, electronically filter the dynamic cardiology publication repository 202 based on recency of publication. In various aspects, the temporal margin 204 can be any suitable electronic data (e.g., one or more scalars, one or more vectors, one or more matrices, one or more tensors, or any suitable combination thereof) that can be considered as defining or demarcating any suitable time window or timespan that is measured backward from a current time or date. If a cardiology publication is published by the dynamic cardiology publication repository 202 within that time window or timespan, such cardiology publication can be considered as being recently published. On the other hand, if a cardiology publication is published by the dynamic cardiology publication repository 202 outside that time window or timespan, such cardiology publication can instead be considered as not being recently published.

As a non-limiting example, the temporal margin 204 can be a 12-hour time window. In such case, any cardiology publication that was published by (e.g., that was added to) the dynamic cardiology publication repository 202 not longer than 12 hours ago (e.g., 12 hours from a current time or date) can be considered as being recent. In contrast, any cardiology publication that was published by (e.g., that was added to) the dynamic cardiology publication repository 202 more than 12 hours ago can, in such case, be considered as not being recent.

As another non-limiting example, the temporal margin 204 can be a 36-hour time window. In such case, any cardiology publication that was published by the dynamic cardiology publication repository 202 not longer than 36 hours ago can be considered as being recent. In contrast, any cardiology publication that was published by the dynamic cardiology publication repository 202 more than 36 hours ago can, in such case, be considered as not being recent.

As even another non-limiting example, the temporal margin 204 can be a 72-hour time window. In such case, any cardiology publication that was published by the dynamic cardiology publication repository 202 not longer than 72 hours ago can be considered as being recent. In contrast, any cardiology publication that was published by the dynamic cardiology publication repository 202 more than 72 hours ago can be considered as not being recent.

As still another non-limiting example, the temporal margin 204 can be a 2-week time window. In such case, any cardiology publication that was published by the dynamic cardiology publication repository 202 not longer than 2 weeks ago can be considered as being recent. In contrast, any cardiology publication that was published by the dynamic cardiology publication repository 202 more than 2 weeks ago can be considered as not being recent.

As yet another non-limiting example, the temporal margin 204 can be a 1-month time window. In such case, any cardiology publication that was published by the dynamic cardiology publication repository 202 not longer than 1 month ago can be considered as being recent. In contrast, any cardiology publication that was published by the dynamic cardiology publication repository 202 more than 1 month ago can be considered as not being recent.

In this way, the retrieval component 118 can leverage the temporal margin 204 to distinguish between recent and non-recent cardiology publications in the dynamic cardiology publication repository 202.

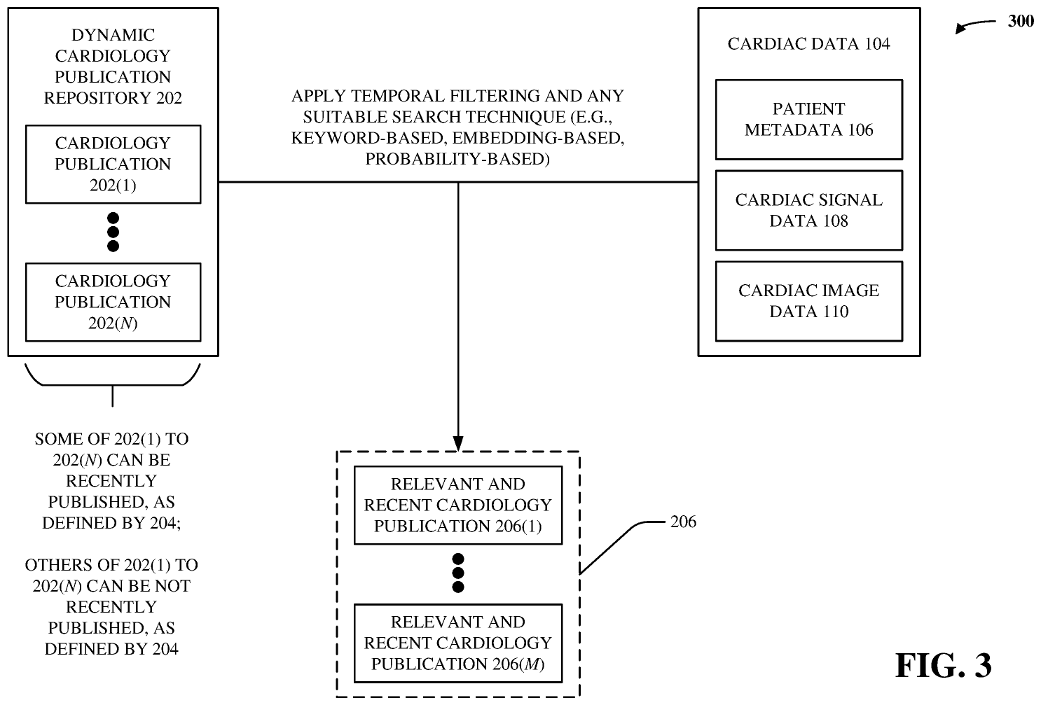

In various aspects, the one or more relevant and recent cardiology publications 206 can be any suitable number of the recent cardiology publications in the dynamic cardiology publication repository 202 that the retrieval component 118 determines are substantively related or pertinent to the cardiac data 104. Non-limiting aspects are described with respect to FIG. 3.

FIG. 3 illustrates an example, non-limiting block diagram 300 showing how the one or more relevant and recent cardiology publications 206 can be identified in the dynamic cardiology publication repository 202 in accordance with one or more embodiments described herein.

In various embodiments, as shown, the dynamic cardiology publication repository 202 can comprise n publications, for any suitable positive integer n: a cardiology publication 202(1) to a cardiology publication 202(n). In various aspects, the cardiology publication 202(1) can be considered as being a first peer-reviewed cardiological research paper or journal article of the dynamic cardiology publication repository 202. Likewise, the cardiology publication 202(n) can be considered as being an n-th peer-reviewed cardiological research paper or journal article of the dynamic cardiology publication repository 202.

Now, in various instances, some of the n cardiology publications in the dynamic cardiology publication repository 202 can be recently published (e.g., can have been added to the dynamic cardiology publication repository 202 within the temporal margin 204 as measured backward from a current time or date). In various cases, others of those n cardiology publications can instead be not recently published (e.g., can instead have been added to the dynamic cardiology publication repository 202 outside of the temporal margin 204 as measured backward from the current time or date). In various aspects, the retrieval component 118 can temporally filter the dynamic cardiology publication repository 202, such that the retrieval component 118 can consider only whichever of those n cardiology publications are considered as recent. Furthermore, in various aspects, the retrieval component 118 can apply any suitable document searching technique to those recently published cardiology publications, so as to identify which (if any) of those recently published cardiology publications are substantively relevant to the cardiac data 104.

As a non-limiting example, the retrieval component 118 can apply any suitable keyword-based document searching technique to the dynamic cardiology publication repository 202. In such cases, the retrieval component 118 can identify (e.g., via named entity recognition) one or more keywords that are contained or recited in the cardiac data 104, and the retrieval component 118 can search the dynamic cardiology publication repository 202 for whichever (if any) recent cardiology publications also contain or recite those one or more keywords. If any given recent cardiology publication contains or recites those one or more keywords, then that given recent cardiology publication can be considered as being substantively relevant or otherwise related to the cardiac data 104. For instance, if the patient metadata 106 recites the keywords “geriatric” and “smoker”, then any recent cardiology publication that also recites the keywords “geriatric” and “smoker” can be considered as being substantively pertinent to the cardiac data 104.

As another non-limiting example, the retrieval component 118 can apply any suitable embedding-based document searching technique to the dynamic cardiology publication repository 202. In such cases, the retrieval component 118 can generate (e.g., via a deep learning autoencoder trained in unsupervised fashion) a first embedding (e.g., a latent vector representation) of the cardiac data 104, the retrieval component 118 can likewise generate or otherwise access second embeddings of respective recent cardiology publications in the dynamic cardiology publication repository 202, and the retrieval component 118 can determine which (if any) of those second embeddings are sufficiently similar (e.g., in terms of Euclidean distance or cosine similarity) to the first embedding. If the second embedding of any given recent cardiology publication is within any suitable threshold level of similarity of the first embedding of the cardiac data 104, then that given recent cardiology publication can be considered as being substantively relevant or otherwise related to the cardiac data 104. Indeed, if the second embedding of that given recent cardiology publication is similar to the first embedding of the cardiac data 104, this can be interpreted to mean that such given recent cardiology publication contains text (e.g., patient metadata), signal measurements (e.g., electrocardiogram traces), or images (e.g., echocardiograms) that are similar to those of the cardiac data 104.

As yet another non-limiting example, the retrieval component 118 can apply any suitable probability-based document searching technique to the dynamic cardiology publication repository 202. For instance, the retrieval component 118 can utilize a Term Frequency-Inverse Document Frequency (TF-IDF) searching technique. As another instance, the retrieval component 118 can utilize a Best Match 25 searching technique.

Regardless of the type of document searching technique that is implemented, the retrieval component 118 can search through whatever recent cardiology publications are in the dynamic cardiology publication repository 202, so as to identify one or more recent publications that are substantively relevant to the cardiac data 104. In various aspects, such recent and substantively relevant publications can be considered as the one or more relevant and recent cardiology publications 206. In various instances, as shown, the one or more relevant and recent cardiology publications 206 can comprise m publications, for any suitable positive integer m≤ n: a relevant and recent cardiology publication 206(1) to a relevant and recent cardiology publication 206 (m). In some cases, the retrieval component 118 can rank (e.g., in keyword-fashion, in embedding-fashion, or in probability-fashion) the recent cardiology publications of the dynamic cardiology publication repository 202 in order of substantive relevance to the cardiac data 104, and whichever m of those recent publications are most highly ranked can be considered as the one or more relevant and recent cardiology publications 206.

Although the herein disclosure has mainly described various embodiments of the retrieval component 118 as first temporally filtering the dynamic cardiology publication repository 202 based on recency and subsequently searching for relevant publications, this is a mere non-limiting example for ease of illustration. In various aspects, the retrieval component 118 can instead first substantively search through the dynamic cardiology publication repository 202 for substantively relevant publications and can subsequently apply temporal filtering based on recency.

Note that the retrieval component 118 can make use of or be assisted, supplemented, or complemented by any suitable text ingestion tools (e.g., software platforms, such as AIRBYTER or APACHE FLUME®, that facilitate the importation, transfer, distribution, or authentication of structured or unstructured textual documents in real-time or in batch fashion) or any suitable text indexing tools (e.g., software platforms, such as DATASHARE® or DOC FETCHER®, that facilitate extraction, processing, or searching of the content of an individual textual document).

In any case, the one or more relevant and recent cardiology publications 206 can be considered as textually describing or explaining recent research, advances, or clinical breakthroughs in the field of cardiology that are pertinent to the cardiac data 104. As a non-limiting example, the cardiac data 104 can pertain to a patient characteristic A, and the one or more relevant and recent cardiology publications 206 can describe or explain a newly-discovered or newly-validated cardiological treatment option for the patient characteristic A. As another non-limiting example, the cardiac data 104 can pertain to a patient characteristic B, and the one or more relevant and recent cardiology publications 206 can describe or explain a newly-discovered or newly-validated cardiological metric that can be computed or derived from echocardiogram images or electrocardiogram traces belonging to the patient characteristic B. As another non-limiting example, the cardiac data 104 can pertain to a patient characteristic C, and the one or more relevant and recent cardiology publications 206 can describe or explain a newly-discovered or newly-validated cardiological prognosis insight for the patient characteristic C.

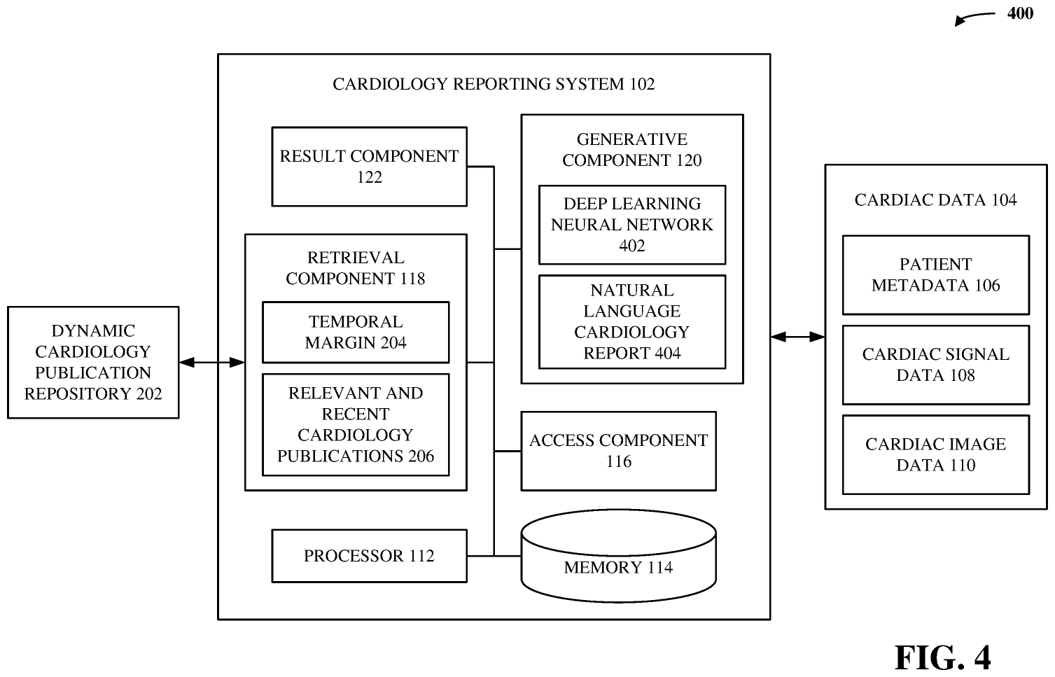

FIG. 4 illustrates a block diagram of an example, non-limiting system 400 including a deep learning neural network and a natural language cardiology report that can facilitate natural language cardiology reporting via retrieval-augmented generative artificial intelligence in accordance with one or more embodiments described herein. As shown, the system 400 can, in some cases, comprise the same components as the system 200, and can further comprise a deep learning neural network 402 and a natural language cardiology report 404.

In various embodiments, the generative component 120 can electronically store, electronically maintain, electronically control, or otherwise electronically access the deep learning neural network 402. In various instances, the deep learning neural network 402 can have or otherwise exhibit any suitable deep learning internal architecture. For instance, the deep learning neural network 402 can have an input layer, one or more hidden layers, and an output layer. In various instances, any of such layers can be coupled together by any suitable interneuron connections or interlayer connections, such as forward connections, skip connections, or recurrent connections. Furthermore, in various cases, any of such layers can be any suitable types of neural network layers having any suitable learnable or trainable internal parameters. For example, any of such input layer, one or more hidden layers, or output layer can be convolutional layers, whose learnable or trainable parameters can be convolutional kernels. As another example, any of such input layer, one or more hidden layers, or output layer can be dense layers, whose learnable or trainable parameters can be weight matrices or bias values. As still another example, any of such input layer, one or more hidden layers, or output layer can be batch normalization layers, whose learnable or trainable parameters can be shift factors or scale factors. As even another example, any of such input layer, one or more hidden layers, or output layer can be LSTM layers, whose learnable or trainable parameters can be input-state weight matrices or hidden-state weight matrices. Further still, in various cases, any of such layers can be any suitable types of neural network layers having any suitable fixed or non-trainable internal parameters. For example, any of such input layer, one or more hidden layers, or output layer can be non-linearity layers, padding layers, pooling layers, or concatenation layers.

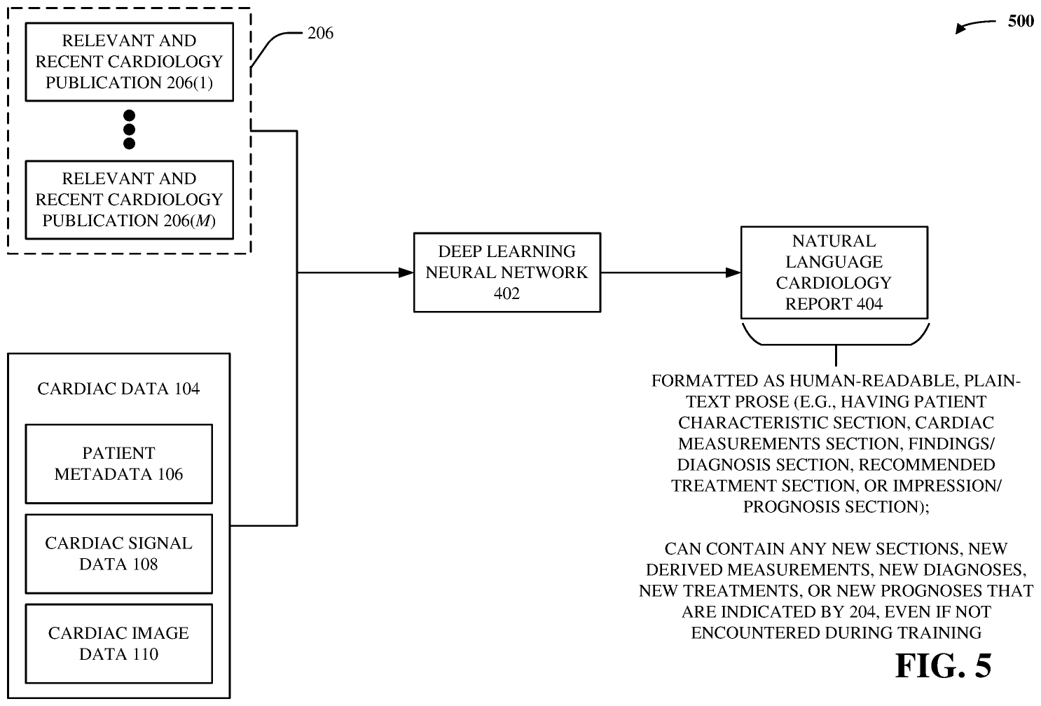

In various aspects, the deep learning neural network 402 can be configured to synthesize cardiology reports based on inputted cardiac data, and based on any suitable electronic documents that accompany such inputted cardiac data. Accordingly, in various instances, the generative component 120 can electronically execute the deep learning neural network 402 on both the cardiac data 104 and on the one or more relevant and recent cardiology publications 206. In various cases, such execution can cause the deep learning neural network 402 to produce the natural language cardiology report 404. Various non-limiting aspects are described with respect to FIG. 5.

FIG. 5 illustrates an example, non-limiting block diagram 500 showing how the natural language cardiology report 404 can be generated in accordance with one or more embodiments described herein.

In various embodiments, the generative component 120 can electronically execute the deep learning neural network 402 on both the cardiac data 104 and the one or more relevant and recent cardiology publications 206. In various aspects, such execution can yield the natural language cardiology report 404. More specifically, the generative component 120 can concatenate the cardiac data 104 and the one or more relevant and recent cardiology publications 206 together. In various instances, the generative component 120 can feed such concatenation to an input layer of the deep learning neural network 402. In various cases, such concatenation can complete a forward pass through one or more hidden layers of the deep learning neural network 402. In various aspects, an output layer of the deep learning neural network 402 can calculate or compute the natural language cardiology report 404, based on activation maps provided by the one or more hidden layers.

In various instances, the natural language cardiology report 404 can be any suitable electronic document that is written in plain text prose and that summarizes, describes, or explains a cardiovascular health of the medical patient.

As a non-limiting example, the natural language cardiology report 404 can comprise a section that describes or explains (e.g., via any suitable sentences, paragraphs, or bullet points) physical characteristics or attributes of the medical patient. Note that the deep learning neural network 402 can synthesize this section based on the patient metadata 106.

As another non-limiting example, the natural language cardiology report 404 can comprise a section that describes or explains (e.g., via any suitable sentences, paragraphs, or bullet points) cardiac measurements or cardiac images of the medical patient. Note that the deep learning neural network 402 can synthesize this section based on the cardiac signal data 108 or the cardiac image data 110.

As yet other non-limiting examples, the natural language cardiology report 404 can comprise: a section that describes or explains a cardiological finding or diagnosis for the medical patient; a section that describes or explains a recommended cardiological treatment for the medical patient; or a section that describes or explains a cardiological impression or prognosis for the medical patient. Note that the deep learning neural network 402 can synthesize these sections by drawing any suitable medical or clinical inferences from the patient metadata 106, from the cardiac signal data 108, or from the cardiac image data 110.

In any case, because the deep learning neural network 402 can receive as input the one or more relevant and recent cardiology publications 206, and because the one or more relevant and recent cardiology publications 206 can describe or explain new or recently-discovered advances in the field of cardiology, the natural language cardiology report 404 can be considered as reflecting such new or recently-discovered cardiological advances, even though such new or recently-discovered cardiological advances may not yet have existed when the deep learning neural network 402 was trained.

Indeed, in various aspects, the deep learning neural network 402 can be or can have been trained to synthesize cardiology reports having various standardized sections. In various cases, it is possible that the one or more relevant and recent cardiology publications 206 describe or explain a new type of section that was just recently standardized. Even though the deep learning neural network 402 was not explicitly trained to synthesize such new type of section, the deep learning neural network 402 can be considered as becoming aware of such new type of section via the one or more relevant and recent cardiology publications 206, and the deep learning neural network 402 can accordingly synthesize the natural language cardiology report 404 so as to include such new type of section. As a non-limiting example, suppose that the deep learning neural network 402 is originally trained to synthesize cardiology reports to have a patient characteristic section, a measurement or image section, a diagnosis section, a treatment section, and a prognosis section. Furthermore, suppose that the one or more relevant and recent cardiology publications 206 describe or explain a new type of section pertaining to cardiac complications arising from COVID-19. It is possible that the deep learning neural network 402 never encountered such a COVID section during its training. However, the deep learning neural network 402 can be considered as becoming aware of such a COVID section due to the one or more relevant and recent cardiology publications 206. Accordingly, the deep learning neural network 402 can cause the natural language cardiology report 404 to have a COVID section pertaining to the medical patient, notwithstanding not having been explicitly trained to synthesize such a COVID section.

Moreover, in various instances, the deep learning neural network 402 can be or can have been trained to synthesize cardiology reports having various standardized cardiological metrics. In various cases, it is possible that the one or more relevant and recent cardiology publications 206 describe or explain a new type of cardiological metric that was just recently standardized. Even though the deep learning neural network 402 was not explicitly trained to synthesize such new type of cardiological metric, the deep learning neural network 402 can be considered as becoming aware of such new type of cardiological metric via the one or more relevant and recent cardiology publications 206, and the deep learning neural network 402 can accordingly synthesize the natural language cardiology report 404 so as to include such new type of cardiological metric. As a non-limiting example, suppose that the deep learning neural network 402 is originally trained to synthesize cardiology reports to have a metrics that can be derived from: pulse rates, blood pressures, blood oxygen concentrations, or electrocardiogram traces. Furthermore, suppose that the one or more relevant and recent cardiology publications 206 describe or explain a new type of cardiological metric that can be derived from the pixel or voxel intensity values of an echocardiogram. It is possible that the deep learning neural network 402 never encountered such an echocardiogram metric during its training. However, the deep learning neural network 402 can be considered as becoming aware of such an echocardiogram metric due to the one or more relevant and recent cardiology publications 206. Accordingly, the deep learning neural network 402 can cause the natural language cardiology report 404 to include an echocardiogram metric pertaining to the medical patient, notwithstanding not having been explicitly trained to synthesize such an echocardiogram metric.

In various aspects, the deep learning neural network 402 can be or can have been trained to synthesize cardiology reports by applying certain standardized cardiological diagnosis insights. In various cases, it is possible that the one or more relevant and recent cardiology publications 206 describe or explain a new type of cardiological diagnosis insight that was just recently standardized or discovered. Even though the deep learning neural network 402 was not explicitly trained to synthesize using such new type of cardiological diagnosis insight, the deep learning neural network 402 can be considered as becoming aware of such new type of cardiological diagnosis insight via the one or more relevant and recent cardiology publications 206, and the deep learning neural network 402 can accordingly synthesize the natural language cardiology report 404 so as to reflect such new type of cardiological diagnosis insight. As a non-limiting example, suppose that the cardiac data 104 indicates a particular patient characteristic and a particularly shaped electrocardiogram trace. Furthermore, suppose that the deep learning neural network 402 was originally trained on cardiological diagnostic insights that held that such characteristic-electrocardiogram combination suggests the presence of a mere cardiac arrhythmia. Further still, suppose that the one or more relevant and recent cardiology publications 206 describe or explain new research or studies that indicate that such characteristic-electrocardiogram combination instead suggests the presence of moderate heart disease rather than a mere cardiac arrhythmia. It is possible that the deep learning neural network 402 never encountered such new cardiological diagnostic insight during its training. However, the deep learning neural network 402 can be considered as becoming aware of such new cardiological diagnostic insight due to the one or more relevant and recent cardiology publications 206. Accordingly, the deep learning neural network 402 can cause the natural language cardiology report 404 to reflect such new cardiological diagnostic insight (e.g., to indicate that the medical patient likely has moderate heart disease rather than a mere cardiac arrhythmia), notwithstanding not having been explicitly trained on it.

In various aspects, the deep learning neural network 402 can be or can have been trained to synthesize cardiology reports by applying certain standardized cardiological treatment insights. In various cases, it is possible that the one or more relevant and recent cardiology publications 206 describe or explain a new type of cardiological treatment insight that was just recently standardized or discovered. Even though the deep learning neural network 402 was not explicitly trained to synthesize using such new type of cardiological treatment insight, the deep learning neural network 402 can be considered as becoming aware of such new type of cardiological treatment insight via the one or more relevant and recent cardiology publications 206, and the deep learning neural network 402 can accordingly synthesize the natural language cardiology report 404 so as to reflect such new type of cardiological treatment insight. As a non-limiting example, suppose that the cardiac data 104 indicates a particular patient characteristic and a particularly shaped cardiac lesion. Furthermore, suppose that the deep learning neural network 402 was originally trained on cardiological treatment insights that held that such characteristic-lesion combination is to be treated with open heart surgery. Further still, suppose that the one or more relevant and recent cardiology publications 206 describe or explain new research or studies that indicate that such characteristic-lesion combination instead should be treated via balloon valvuloplasty. It is possible that the deep learning neural network 402 never encountered such new cardiological treatment insight during its training. However, the deep learning neural network 402 can be considered as becoming aware of such new cardiological treatment insight due to the one or more relevant and recent cardiology publications 206. Accordingly, the deep learning neural network 402 can cause the natural language cardiology report 404 to reflect such new cardiological treatment insight (e.g., to indicate that the medical patient should be treated via balloon valvuloplasty rather than via open heart surgery), notwithstanding not having been explicitly trained on it.

In various instances, the deep learning neural network 402 can be or can have been trained to synthesize cardiology reports by applying certain standardized cardiological prognosis insights. In various cases, it is possible that the one or more relevant and recent cardiology publications 206 describe or explain a new type of cardiological prognosis insight that was just recently standardized or discovered. Even though the deep learning neural network 402 was not explicitly trained to synthesize using such new type of cardiological prognosis insight, the deep learning neural network 402 can be considered as becoming aware of such new type of cardiological prognosis insight via the one or more relevant and recent cardiology publications 206, and the deep learning neural network 402 can accordingly synthesize the natural language cardiology report 404 so as to reflect such new type of cardiological prognosis insight. As a non-limiting example, suppose that the cardiac data 104 indicates a particular patient characteristic and a particularly shaped phonocardiogram trace. Furthermore, suppose that the deep learning neural network 402 was originally trained on cardiological prognosis insights that held that such characteristic-phonocardiogram combination resolves after about two months. Further still, suppose that the one or more relevant and recent cardiology publications 206 describe or explain new research or studies that indicate that such characteristic-phonocardiogram combination instead resolves after about five weeks. It is possible that the deep learning neural network 402 never encountered such new cardiological prognosis insight during its training. However, the deep learning neural network 402 can be considered as becoming aware of such new cardiological prognosis insight due to the one or more relevant and recent cardiology publications 206. Accordingly, the deep learning neural network 402 can cause the natural language cardiology report 404 to reflect such new cardiological prognosis insight (e.g., to indicate that the medical patient's ailments are likely to resolve after about five weeks instead of two months), notwithstanding not having been explicitly trained on it.

In this way, the deep learning neural network 402 can be able to synthesize the natural language cardiology report 404 consistently with the latest, cutting-edge cardiological advancements indicated by the one or more relevant and recent cardiology publications 206.

In various embodiments, the result component 122 can electronically initiate or perform any suitable electronic actions, based on the natural language cardiology report 404. Indeed, in various aspects, the result component 122 can electronically render the natural language cardiology report 404 on any suitable electronic display or computer screen. Accordingly, a medical professional (e.g., a physician, a nurse) who is attending to the medical patient can visually see, read, or view the natural language cardiology report 404. In various instances, the result component 122 can electronically prompt (e.g., via the electronic display or computer screen) the medical professional to approve or disapprove of the natural language cardiology report 404. If the medical professional provides input (e.g., via any suitable human-computer interface device such as a keyboard, keypad, or touchscreen) that disapproves of the natural language cardiology report 404, the result component 122 can instruct the retrieval component 118 to search the dynamic cardiology publication repository 202 for different cardiology publications that are recent and relevant, and the result component 122 can instruct the generative component 120 to generate a new iteration of the natural language cardiology report 404 based on those new recent and relevant cardiology publications. In some cases, if the medical professional provides input that approves of the natural language cardiology report 404, the result component 122 can electronically transmit the natural language cardiology report 404 to any other suitable computing device for the performance of any suitable downstream computing task or analysis. As a non-limiting example, the result component 122 can electronically transmit the natural language cardiology report 404 to a picture archiving and communication system (PACS), and the PACS can electronically add, insert, or otherwise append the natural language cardiology report 404 to an electronic medical record or electronic medical history associated with the medical patient. As another non-limiting example, the result component 122 can electronically transmit the natural language cardiology report 404 to a hospital billing system, and the hospital billing system can electronically compute or estimate (e.g., such as via machine learning) a medical bill for the medical patient, based on the content of the natural language cardiology report 404.

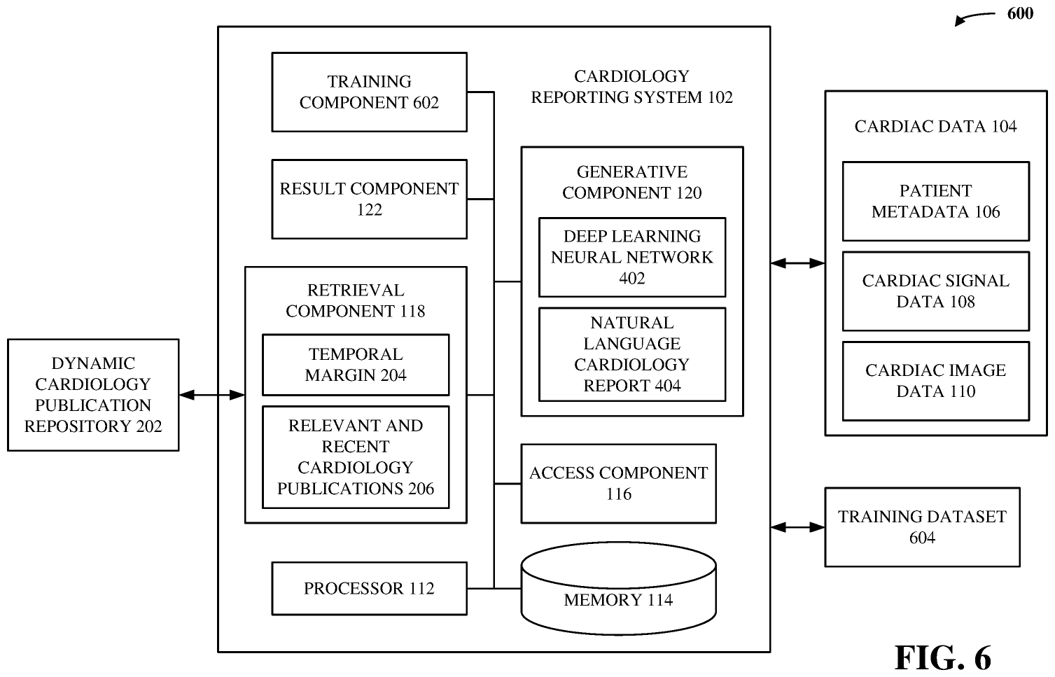

In order for the natural language cardiology report 404 to be accurate, correct, or reliable, the deep learning neural network 402 can first undergo training, as described with respect to FIGS. 6-8.

FIG. 6 illustrates a block diagram of an example, non-limiting system 600 including a training component and a training dataset that can facilitate natural language cardiology reporting via retrieval-augmented generative artificial intelligence in accordance with one or more embodiments described herein. As shown, the system 600 can, in some cases, comprise the same components as the system 400, and can further comprise a training component 602 and a training dataset 604.

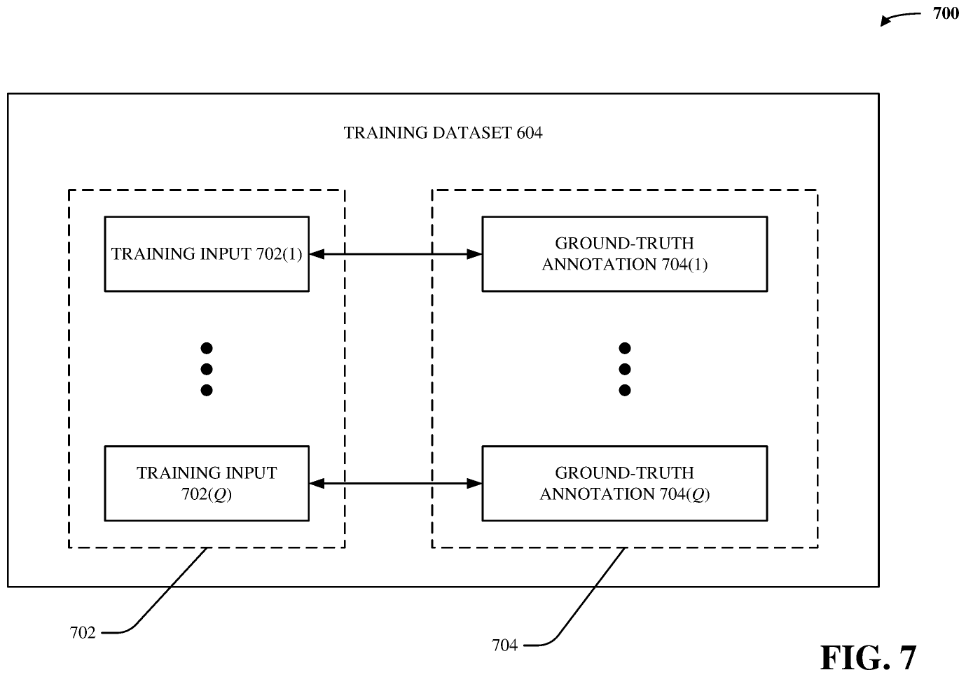

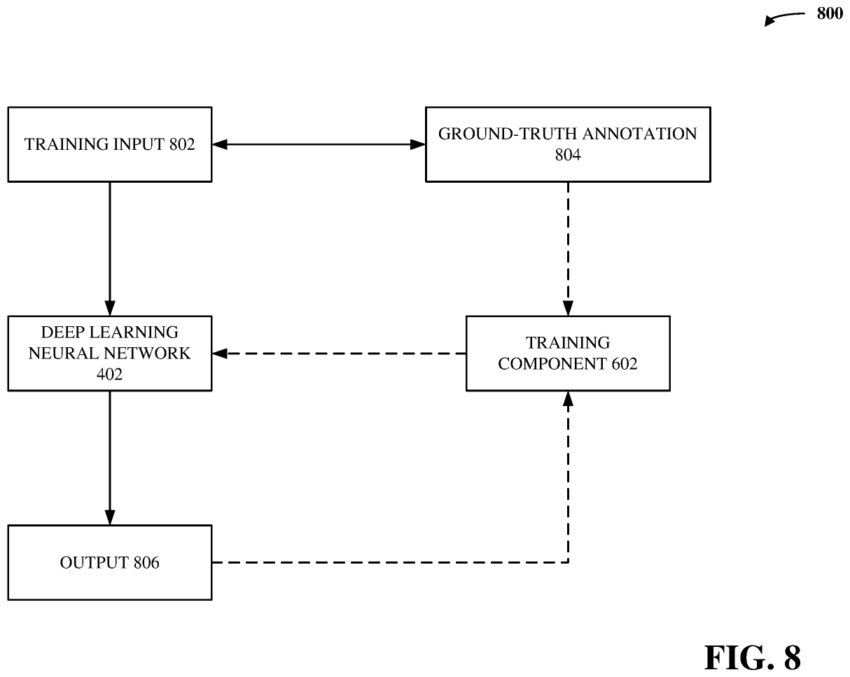

In various aspects, the access component 116 can electronically receive, retrieve, or otherwise access, from any suitable source, the training dataset 604, and the training component 602 can electronically train the deep learning neural network 402 on the training dataset 604. Non-limiting aspects are described with respect to FIGS. 7-8.

FIG. 7 illustrates an example, non-limiting block diagram 700 of the training dataset 604 in accordance with one or more embodiments described herein.

In various aspects, the training dataset 604 can comprise a set of training inputs 702. In various instances, the set of training inputs 702 can include q inputs for any suitable positive integer q: a training input 702(1) to a training input 702(q). In various instances, each of the set of training inputs 702 can be training cardiac data having the same format, size, or dimensionality as the cardiac data 104. In various other instances, each of the set of training inputs 702 can be a concatenation between training cardiac data (e.g., having the same format, size, or dimensionality as the cardiac data 104) and one or more training cardiology publications (e.g., having the same format, size, or dimensionality as the one or more relevant and recent cardiology publications 206). In even other instances, each of the set of training inputs 702 can be a concatenation between training cardiac data (e.g., having the same format, size, or dimensionality as the cardiac data 104) and dummy variables (e.g., being blank but having the same format, size, or dimensionality as the one or more relevant and recent cardiology publications 206).

In various aspects, the training dataset 604 can comprise a set of ground-truth annotations 704 that can respectively correspond to the set of training inputs 702. Accordingly, since the set of training inputs 702 can have q inputs, the set of ground-truth annotations 704 can have q annotations: a ground-truth annotation 704(1) to a ground-truth annotation 704(q). In various instances, each of the set of ground-truth annotations 704 can be a correct or accurate natural language cardiology report (e.g., having the same format, size, or dimensionality as the natural language cardiology report 404) that is known or deemed to correspond to a respective one of the set of training inputs 702. As a non-limiting example, the ground-truth annotation 704(1) can correspond to the training input 702(1). Accordingly, the ground-truth annotation 704(1) can be the correct or accurate natural language cardiology report that is known or deemed to correspond to the training input 702(1). As another non-limiting example, the ground-truth annotation 704(q) can correspond to the training input 702(q). Accordingly, the ground-truth annotation 704(q) can be the correct or accurate natural language cardiology report that is known or deemed to correspond to the training input 702(q).

FIG. 8 illustrates an example, non-limiting block diagram 800 showing how the deep learning neural network 402 can be trained on the training dataset 604 in accordance with one or more embodiments described herein.

In various aspects, prior to beginning training, trainable internal parameters (e.g., convolutional kernels, weight matrices, bias values) of the deep learning neural network 402 can be initialized in any suitable fashion (e.g., via random initialization).

In various aspects, the training component 602 can select a training input 802 and a ground-truth annotation 804 corresponding to the training input 802 from the training dataset 604. In various instances, the training component 602 can execute the deep learning neural network 402 on the training input 802, thereby causing the deep learning neural network 402 to produce an output 806. More specifically, in some cases, the training component 602 can feed the training input 802 to the input layer of the deep learning neural network 402, the training input 802 can complete a forward pass through the one or more hidden layers of the deep learning neural network 402, and the output layer of the deep learning neural network 402 can compute the output 806 based on activation maps or feature maps provided by the one or more hidden layers of the deep learning neural network 402.

Note that the format, size, or dimensionality of the output 806 can be dictated by the number, arrangement, sizes, or other characteristics of the neurons, convolutional kernels, or other internal parameters of the output layer (or of any other layers) of the deep learning neural network 402. Accordingly, the output 806 can be forced to have any desired format, size, or dimensionality, by adding, removing, or otherwise adjusting characteristics of the output layer (or of any other layers) of the deep learning neural network 402. So, the output 806 can be considered as the predicted or inferred natural language cardiology report that the deep learning neural network 402 believes should correspond to the training input 802. In contrast, the ground-truth annotation 804 can be considered as the correct or accurate natural language cardiology report that is known or deemed to correspond to the training input 802. Note that, if the deep learning neural network 402 has so far undergone no or little training, then the output 806 can be highly inaccurate (e.g., can appear to be gibberish). In other words, the output 806 can be very different from the ground-truth annotation 804.

In various aspects, the training component 602 can compute any suitable error or loss (e.g., mean absolute error, mean squared error, cross-entropy error) between the output 806 and the ground-truth annotation 804. In various instances, the training component 602 can incrementally update the trainable internal parameters of the deep learning neural network 402, via backpropagation (e.g., stochastic gradient descent) driven by the computed error or loss.

In various cases, such execution-and-update procedure can be repeated for any suitable number of training inputs (e.g., for each training input in the training dataset 604). This can ultimately cause the trainable internal parameters of the deep learning neural network 402 to become iteratively optimized for accurately generating natural language cardiology reports based on inputted cardiac data (which inputted cardiac data may or may not be accompanied by recent and relevant cardiology publications). In various aspects, the training component 602 can implement any suitable training batch sizes, any suitable error, loss, or objective functions, or any suitable training termination criteria.

Although the above description mainly describes the deep learning neural network 402 as being trained in supervised fashion, this is a mere non-limiting example for ease of illustration and explanation. In various cases, the training component 602 can implement any other suitable training paradigms (e.g., unsupervised training, reinforcement learning) to train the deep learning neural network 402.

Although the herein disclosure has mainly described various embodiments in which the temporal margin 204 is a fixed time window (e.g., 12-hour time window, 1-week time window), this is a mere non-limiting example for ease of explanation. In various other embodiments, the temporal margin 204 can be any suitable variable time window. As a non-limiting example, the temporal margin 204 can, in various cases, be whatever amount of time has passed or elapsed since a most recent training session of the deep learning neural network 402. In such cases, the retrieval component 118 can be considered as searching the dynamic cardiology publication repository 202 for publications that are relevant to the cardiac data 104 and that were published after the deep learning neural network 402 was most recently trained, retrained, or fine-tuned. In other words, the most recent training session of the deep learning neural network 402 can have occurred at a known past time or date, and the retrieval component 118 can search the dynamic cardiology publication repository 202 only for publications that were added or shared after that known past time or date. This can help to guarantee that the cardiac data 104 is accompanied by cardiology research findings or advances that the deep learning neural network 402 did not encounter during training.

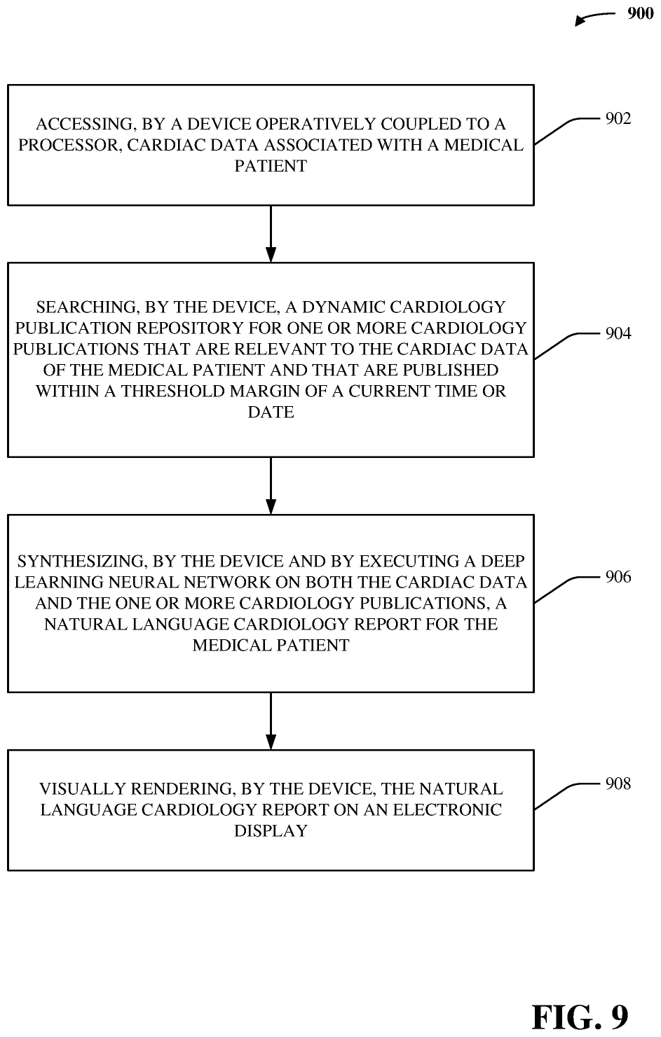

FIG. 9 illustrates a flow diagram of an example, non-limiting computer-implemented method 900 that can facilitate natural language cardiology reporting via retrieval-augmented generative artificial intelligence in accordance with one or more embodiments described herein. In various cases, the cardiology reporting system 102 can facilitate the computer-implemented method 900.

In various embodiments, act 902 can include accessing, by a device (e.g., via 116) operatively coupled to a processor (e.g., 112), cardiac data (e.g., 104) associated with a medical patient.

In various aspects, act 904 can include searching, by the device (e.g., via 118), a dynamic cardiology publication repository (e.g., 202) for one or more cardiology publications (e.g., 206) that are relevant to the cardiac data of the medical patient and that are published within a threshold margin (e.g., 204) of a current time or date.

In various instances, act 906 can include synthesizing, by the device (e.g., via 120) and by executing a deep learning neural network (e.g., 402) on both the cardiac data and the one or more cardiology publications, a natural language cardiology report (e.g., 404) for the medical patient.

In various cases, act 908 can include visually rendering, by the device (e.g., via 122), the natural language cardiology report on an electronic display.

Although not explicitly shown in FIG. 9, the dynamic cardiology publication repository can be a medical website, and the one or more cardiology publications can be cardiology research papers or cardiology journal articles published by the medical website.

Although not explicitly shown in FIG. 9, the cardiac data can comprise: one or more computed tomography images (e.g., 110) of a heart of the medical patient; one or more magnetic resonance images (e.g., 110) of the heart of the medical patient; one or more echocardiogram images (e.g., 110) of the heart of the medical patient; or one or more positron emission tomography images (e.g., 110) of the heart of the medical patient.

Although not explicitly shown in FIG. 9, the cardiac data can comprise: an electrocardiogram (e.g., 108) of the medical patient; a photoplethysmogram (e.g., 108) of the medical patient; a seismocardiogram (e.g., 108) of the medical patient; or a phonocardiogram (e.g., 108) of the medical patient.

Although not explicitly shown in FIG. 9, the cardiac data can comprise physical characteristics or attributes (e.g., 106) of the medical patient.

Although not explicitly shown in FIG. 9, the device can search the dynamic cardiology publication repository via: a keyword search technique; an embedding search technique; or a probabilistic information retrieval search technique.

Although not explicitly shown in FIG. 9, the computer-implemented method 900 can comprise: prompting, by the device (e.g., via 122) and on the electronic display, a user to accept or reject the natural language cardiology report.

Although the herein disclosure describes various embodiments as applying to natural language cardiology reporting, this is a mere non-limiting example for ease of illustration and explanation. In various aspects, various embodiments can be applied or extrapolated to any suitable natural language medical reporting (e.g., not limited just to the medical field of cardiology). In such cases, the cardiac data 104 can be more generally considered as medical data, the cardiac signal data 108 can be more generally considered as medical signal data (e.g., breathing rate, body temperature), the cardiac image data 110 can be more generally considered as medical image data (e.g., medical scanned images of any suitable anatomical structures), and the dynamic cardiology publication repository 202 can be more generally considered as a dynamic medical publication repository.

Indeed, various embodiments can include a computer program product for facilitating natural language medical reporting via retrieval-augmented generative artificial intelligence. In various aspects, the computer program product can comprise a non-transitory computer-readable memory (e.g., 114) having program instructions embodied therewith. In various instances, the program instructions can be executable by a processor (e.g., 112) to cause the processor to: access medical data (e.g., 104) associated with a medical patient; search a dynamic medical publication repository (e.g., 202) for one or more medical publications (e.g., 206) that are relevant to the medical data of the medical patient and that are published within a threshold margin (e.g., 204) of a current time or date; and synthesize a natural language medical report (e.g., 404) for the medical patient, by executing a deep learning neural network (e.g., 402) on both the medical data and the one or more medical publications. In various instances, the dynamic medical publication repository can be a medical website, and the one or more medical publications can be medical research papers or medical journal articles published by the medical website. In various cases, the medical data can comprise: a body temperature of the medical patient; a pulse rate of the medical patient; a respiration rate of the medical patient; or a blood pressure of the medical patient. In various aspects, the medical data can comprise a medical scanned image of the medical patient.

In various instances, machine learning algorithms or models can be implemented in any suitable way to facilitate any suitable aspects described herein. To facilitate some of the above-described machine learning aspects of various embodiments, consider the following discussion of artificial intelligence (AI). Various embodiments described herein can employ artificial intelligence to facilitate automating one or more features or functionalities. The components can employ various AI-based schemes for carrying out various embodiments/examples disclosed herein. In order to provide for or aid in the numerous determinations (e.g., determine, ascertain, infer, calculate, predict, prognose, estimate, derive, forecast, detect, compute) described herein, components described herein can examine the entirety or a subset of the data to which it is granted access and can provide for reasoning about or determine states of the system or environment from a set of observations as captured via events or data. Determinations can be employed to identify a specific context or action, or can generate a probability distribution over states, for example. The determinations can be probabilistic; that is, the computation of a probability distribution over states of interest based on a consideration of data and events. Determinations can also refer to techniques employed for composing higher-level events from a set of events or data.

Such determinations can result in the construction of new events or actions from a set of observed events or stored event data, whether or not the events are correlated in close temporal proximity, and whether the events and data come from one or several event and data sources. Components disclosed herein can employ various classification (explicitly trained (e.g., via training data) as well as implicitly trained (e.g., via observing behavior, preferences, historical information, receiving extrinsic information, and so on)) schemes or systems (e.g., support vector machines, neural networks, expert systems, Bayesian belief networks, fuzzy logic, data fusion engines, and so on) in connection with performing automatic or determined action in connection with the claimed subject matter. Thus, classification schemes or systems can be used to automatically learn and perform a number of functions, actions, or determinations.

A classifier can map an input attribute vector, z=(z₁, z₂, z₃, z₄, zₙ), to a confidence that the input belongs to a class, as by f(z)=confidence (class). Such classification can employ a probabilistic or statistical-based analysis (e.g., factoring into the analysis utilities and costs) to determinate an action to be automatically performed. A support vector machine (SVM) can be an example of a classifier that can be employed. The SVM operates by finding a hyper-surface in the space of possible inputs, where the hyper-surface attempts to split the triggering criteria from the non-triggering events. Intuitively, this makes the classification correct for testing data that is near, but not identical to training data. Other directed and undirected model classification approaches include, e.g., naïve Bayes, Bayesian networks, decision trees, neural networks, fuzzy logic models, or probabilistic classification models providing different patterns of independence, any of which can be employed. Classification as used herein also is inclusive of statistical regression that is utilized to develop models of priority.

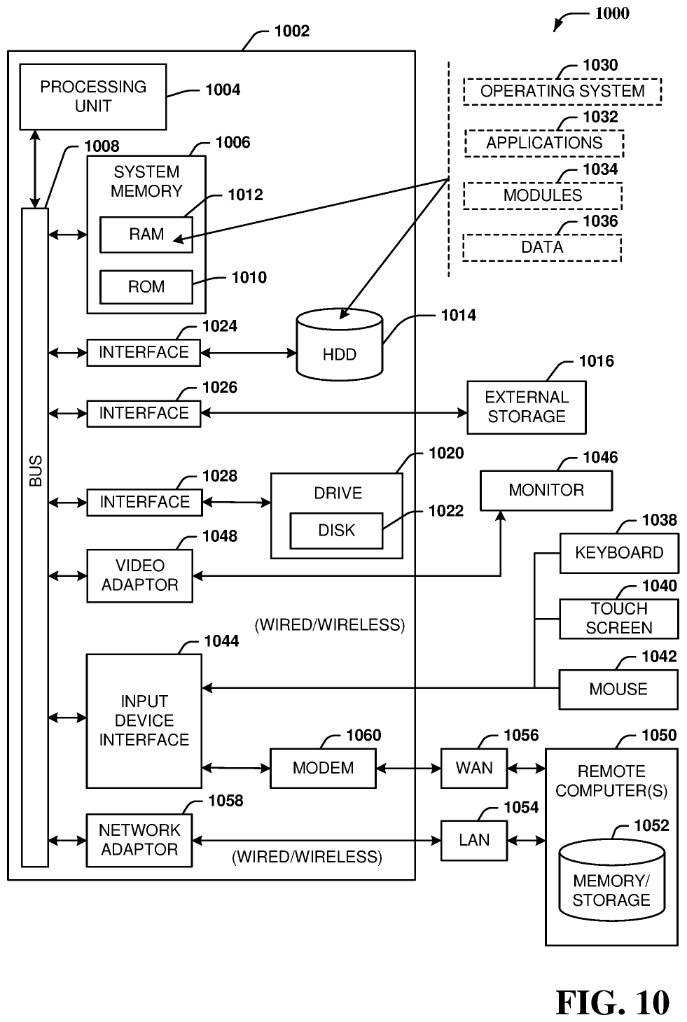

In order to provide additional context for various embodiments described herein, FIG. 10 and the following discussion are intended to provide a brief, general description of a suitable computing environment 1000 in which the various embodiments of the embodiment described herein can be implemented. While the embodiments have been described above in the general context of computer-executable instructions that can run on one or more computers, those skilled in the art will recognize that the embodiments can be also implemented in combination with other program modules or as a combination of hardware and software.

Generally, program modules include routines, programs, components, data structures, etc., that perform particular tasks or implement particular abstract data types. Moreover, those skilled in the art will appreciate that the inventive methods can be practiced with other computer system configurations, including single-processor or multi-processor computer systems, minicomputers, mainframe computers, Internet of Things (IoT) devices, distributed computing systems, as well as personal computers, hand-held computing devices, microprocessor-based or programmable consumer electronics, and the like, each of which can be operatively coupled to one or more associated devices.

The illustrated embodiments of the embodiments herein can be also practiced in distributed computing environments where certain tasks are performed by remote processing devices that are linked through a communications network. In a distributed computing environment, program modules can be located in both local and remote memory storage devices.

Computing devices typically include a variety of media, which can include computer-readable storage media, machine-readable storage media, or communications media, which two terms are used herein differently from one another as follows. Computer-readable storage media or machine-readable storage media can be any available storage media that can be accessed by the computer and includes both volatile and nonvolatile media, removable and non-removable media. By way of example, and not limitation, computer-readable storage media or machine-readable storage media can be implemented in connection with any method or technology for storage of information such as computer-readable or machine-readable instructions, program modules, structured data or unstructured data.

Computer-readable storage media can include, but are not limited to, random access memory (RAM), read only memory (ROM), electrically erasable programmable read only memory (EEPROM), flash memory or other memory technology, compact disk read only memory (CD-ROM), digital versatile disk (DVD), Blu-ray disc (BD) or other optical disk storage, magnetic cassettes, magnetic tape, magnetic disk storage or other magnetic storage devices, solid state drives or other solid state storage devices, or other tangible or non-transitory media which can be used to store desired information. In this regard, the terms “tangible” or “non-transitory” herein as applied to storage, memory or computer-readable media, are to be understood to exclude only propagating transitory signals per se as modifiers and do not relinquish rights to all standard storage, memory or computer-readable media that are not only propagating transitory signals per se.

Computer-readable storage media can be accessed by one or more local or remote computing devices, e.g., via access requests, queries or other data retrieval protocols, for a variety of operations with respect to the information stored by the medium.

Communications media typically embody computer-readable instructions, data structures, program modules or other structured or unstructured data in a data signal such as a modulated data signal, e.g., a carrier wave or other transport mechanism, and includes any information delivery or transport media. The term “modulated data signal” or signals refers to a signal that has one or more of its characteristics set or changed in such a manner as to encode information in one or more signals. By way of example, and not limitation, communication media include wired media, such as a wired network or direct-wired connection, and wireless media such as acoustic, RF, infrared and other wireless media.

With reference again to FIG. 10, the example environment 1000 for implementing various embodiments of the aspects described herein includes a computer 1002, the computer 1002 including a processing unit 1004, a system memory 1006 and a system bus 1008. The system bus 1008 couples system components including, but not limited to, the system memory 1006 to the processing unit 1004. The processing unit 1004 can be any of various commercially available processors. Dual microprocessors and other multi-processor architectures can also be employed as the processing unit 1004.

The system bus 1008 can be any of several types of bus structure that can further interconnect to a memory bus (with or without a memory controller), a peripheral bus, and a local bus using any of a variety of commercially available bus architectures. The system memory 1006 includes ROM 1010 and RAM 1012. A basic input/output system (BIOS) can be stored in a non-volatile memory such as ROM, erasable programmable read only memory (EPROM), EEPROM, which BIOS contains the basic routines that help to transfer information between elements within the computer 1002, such as during startup. The RAM 1012 can also include a high-speed RAM such as static RAM for caching data.

The computer 1002 further includes an internal hard disk drive (HDD) 1014 (e.g., EIDE, SATA), one or more external storage devices 1016 (e.g., a magnetic floppy disk drive (FDD) 1016, a memory stick or flash drive reader, a memory card reader, etc.) and a drive 1020, e.g., such as a solid state drive, an optical disk drive, which can read or write from a disk 1022, such as a CD-ROM disc, a DVD, a BD, etc. Alternatively, where a solid state drive is involved, disk 1022 would not be included, unless separate. While the internal HDD 1014 is illustrated as located within the computer 1002, the internal HDD 1014 can also be configured for external use in a suitable chassis (not shown). Additionally, while not shown in environment 1000, a solid state drive (SSD) could be used in addition to, or in place of, an HDD 1014. The HDD 1014, external storage device(s) 1016 and drive 1020 can be connected to the system bus 1008 by an HDD interface 1024, an external storage interface 1026 and a drive interface 1028, respectively. The interface 1024 for external drive implementations can include at least one or both of Universal Serial Bus (USB) and Institute of Electrical and Electronics Engineers (IEEE) 1394 interface technologies. Other external drive connection technologies are within contemplation of the embodiments described herein.

The drives and their associated computer-readable storage media provide nonvolatile storage of data, data structures, computer-executable instructions, and so forth. For the computer 1002, the drives and storage media accommodate the storage of any data in a suitable digital format. Although the description of computer-readable storage media above refers to respective types of storage devices, it should be appreciated by those skilled in the art that other types of storage media which are readable by a computer, whether presently existing or developed in the future, could also be used in the example operating environment, and further, that any such storage media can contain computer-executable instructions for performing the methods described herein.

A number of program modules can be stored in the drives and RAM 1012, including an operating system 1030, one or more application programs 1032, other program modules 1034 and program data 1036. All or portions of the operating system, applications, modules, or data can also be cached in the RAM 1012. The systems and methods described herein can be implemented utilizing various commercially available operating systems or combinations of operating systems.

Computer 1002 can optionally comprise emulation technologies. For example, a hypervisor (not shown) or other intermediary can emulate a hardware environment for operating system 1030, and the emulated hardware can optionally be different from the hardware illustrated in FIG. 10. In such an embodiment, operating system 1030 can comprise one virtual machine (VM) of multiple VMs hosted at computer 1002. Furthermore, operating system 1030 can provide runtime environments, such as the Java runtime environment or the .NET framework, for applications 1032. Runtime environments are consistent execution environments that allow applications 1032 to run on any operating system that includes the runtime environment. Similarly, operating system 1030 can support containers, and applications 1032 can be in the form of containers, which are lightweight, standalone, executable packages of software that include, e.g., code, runtime, system tools, system libraries and settings for an application.

Further, computer 1002 can be enable with a security module, such as a trusted processing module (TPM). For instance with a TPM, boot components hash next in time boot components, and wait for a match of results to secured values, before loading a next boot component. This process can take place at any layer in the code execution stack of computer 1002, e.g., applied at the application execution level or at the operating system (OS) kernel level, thereby enabling security at any level of code execution.

A user can enter commands and information into the computer 1002 through one or more wired/wireless input devices, e.g., a keyboard 1038, a touch screen 1040, and a pointing device, such as a mouse 1042. Other input devices (not shown) can include a microphone, an infrared (IR) remote control, a radio frequency (RF) remote control, or other remote control, a joystick, a virtual reality controller or virtual reality headset, a game pad, a stylus pen, an image input device, e.g., camera(s), a gesture sensor input device, a vision movement sensor input device, an emotion or facial detection device, a biometric input device, e.g., fingerprint or iris scanner, or the like. These and other input devices are often connected to the processing unit 1004 through an input device interface 1044 that can be coupled to the system bus 1008, but can be connected by other interfaces, such as a parallel port, an IEEE 1394 serial port, a game port, a USB port, an IR interface, a BLUETOOTH® interface, etc.

A monitor 1046 or other type of display device can be also connected to the system bus 1008 via an interface, such as a video adapter 1048. In addition to the monitor 1046, a computer typically includes other peripheral output devices (not shown), such as speakers, printers, etc.

The computer 1002 can operate in a networked environment using logical connections via wired or wireless communications to one or more remote computers, such as a remote computer(s) 1050. The remote computer(s) 1050 can be a workstation, a server computer, a router, a personal computer, portable computer, microprocessor-based entertainment appliance, a peer device or other common network node, and typically includes many or all of the elements described relative to the computer 1002, although, for purposes of brevity, only a memory/storage device 1052 is illustrated. The logical connections depicted include wired/wireless connectivity to a local area network (LAN) 1054 or larger networks, e.g., a wide area network (WAN) 1056. Such LAN and WAN networking environments are commonplace in offices and companies, and facilitate enterprise-wide computer networks, such as intranets, all of which can connect to a global communications network, e.g., the Internet.

When used in a LAN networking environment, the computer 1002 can be connected to the local network 1054 through a wired or wireless communication network interface or adapter 1058. The adapter 1058 can facilitate wired or wireless communication to the LAN 1054, which can also include a wireless access point (AP) disposed thereon for communicating with the adapter 1058 in a wireless mode.

When used in a WAN networking environment, the computer 1002 can include a modem 1060 or can be connected to a communications server on the WAN 1056 via other means for establishing communications over the WAN 1056, such as by way of the Internet. The modem 1060, which can be internal or external and a wired or wireless device, can be connected to the system bus 1008 via the input device interface 1044. In a networked environment, program modules depicted relative to the computer 1002 or portions thereof, can be stored in the remote memory/storage device 1052. It will be appreciated that the network connections shown are example and other means of establishing a communications link between the computers can be used.

When used in either a LAN or WAN networking environment, the computer 1002 can access cloud storage systems or other network-based storage systems in addition to, or in place of, external storage devices 1016 as described above, such as but not limited to a network virtual machine providing one or more aspects of storage or processing of information. Generally, a connection between the computer 1002 and a cloud storage system can be established over a LAN 1054 or WAN 1056 e.g., by the adapter 1058 or modem 1060, respectively. Upon connecting the computer 1002 to an associated cloud storage system, the external storage interface 1026 can, with the aid of the adapter 1058 or modem 1060, manage storage provided by the cloud storage system as it would other types of external storage. For instance, the external storage interface 1026 can be configured to provide access to cloud storage sources as if those sources were physically connected to the computer 1002.

The computer 1002 can be operable to communicate with any wireless devices or entities operatively disposed in wireless communication, e.g., a printer, scanner, desktop or portable computer, portable data assistant, communications satellite, any piece of equipment or location associated with a wirelessly detectable tag (e.g., a kiosk, news stand, store shelf, etc.), and telephone. This can include Wireless Fidelity (Wi-Fi) and BLUETOOTH® wireless technologies. Thus, the communication can be a predefined structure as with a conventional network or simply an ad hoc communication between at least two devices.

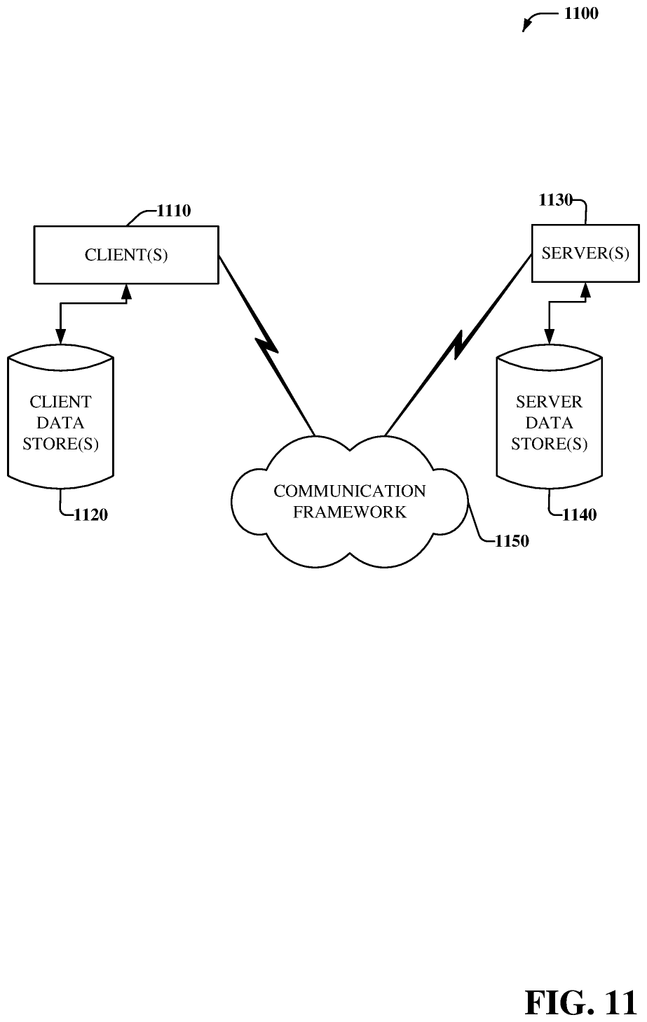

FIG. 11 is a schematic block diagram of a sample computing environment 1100 with which the disclosed subject matter can interact. The sample computing environment 1100 includes one or more client(s) 1110. The client(s) 1110 can be hardware or software (e.g., threads, processes, computing devices). The sample computing environment 1100 also includes one or more server(s) 1130. The server(s) 1130 can also be hardware or software (e.g., threads, processes, computing devices). The servers 1130 can house threads to perform transformations by employing one or more embodiments as described herein, for example. One possible communication between a client 1110 and a server 1130 can be in the form of a data packet adapted to be transmitted between two or more computer processes. The sample computing environment 1100 includes a communication framework 1150 that can be employed to facilitate communications between the client(s) 1110 and the server(s) 1130. The client(s) 1110 are operably connected to one or more client data store(s) 1120 that can be employed to store information local to the client(s) 1110. Similarly, the server(s) 1130 are operably connected to one or more server data store(s) 1140 that can be employed to store information local to the servers 1130.

Various embodiments may be a system, a method, an apparatus or a computer program product at any possible technical detail level of integration. The computer program product can include a computer readable storage medium (or media) having computer readable program instructions thereon for causing a processor to carry out aspects of various embodiments. The computer readable storage medium can be a tangible device that can retain and store instructions for use by an instruction execution device. The computer readable storage medium can be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium can also include the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network or a wireless network. The network can comprise copper transmission cables, optical transmission fibers, wireless transmission, routers, firewalls, switches, gateway computers or edge servers. A network adapter card or network interface in each computing/processing device receives computer readable program instructions from the network and forwards the computer readable program instructions for storage in a computer readable storage medium within the respective computing/processing device. Computer readable program instructions for carrying out operations of various embodiments can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions can execute entirely on the user's computer, partly on the user's computer, as a stand-alone software package, partly on the user's computer and partly on a remote computer or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform various aspects.

Various aspects are described herein with reference to flowchart illustrations or block diagrams of methods, apparatus (systems), and computer program products according to various embodiments. It will be understood that each block of the flowchart illustrations or block diagrams, and combinations of blocks in the flowchart illustrations or block diagrams, can be implemented by computer readable program instructions. These computer readable program instructions can be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart or block diagram block or blocks. These computer readable program instructions can also be stored in a computer readable storage medium that can direct a computer, a programmable data processing apparatus, or other devices to function in a particular manner, such that the computer readable storage medium having instructions stored therein comprises an article of manufacture including instructions which implement aspects of the function/act specified in the flowchart or block diagram block or blocks. The computer readable program instructions can also be loaded onto a computer, other programmable data processing apparatus, or other device to cause a series of operational acts to be performed on the computer, other programmable apparatus or other device to produce a computer implemented process, such that the instructions which execute on the computer, other programmable apparatus, or other device implement the functions/acts specified in the flowchart or block diagram block or blocks.

The flowcharts and block diagrams in the Figures illustrate the architecture, functionality, and operation of possible implementations of systems, methods, and computer program products according to various embodiments. In this regard, each block in the flowchart or block diagrams can represent a module, segment, or portion of instructions, which comprises one or more executable instructions for implementing the specified logical function(s). In some alternative implementations, the functions noted in the blocks can occur out of the order noted in the Figures. For example, two blocks shown in succession can, in fact, be executed substantially concurrently, or the blocks can sometimes be executed in the reverse order, depending upon the functionality involved. It will also be noted that each block of the block diagrams or flowchart illustration, and combinations of blocks in the block diagrams or flowchart illustration, can be implemented by special purpose hardware-based systems that perform the specified functions or acts or carry out combinations of special purpose hardware and computer instructions.

While the subject matter has been described above in the general context of computer-executable instructions of a computer program product that runs on a computer or computers, those skilled in the art will recognize that this disclosure also can or can be implemented in combination with other program modules. Generally, program modules include routines, programs, components, data structures, etc. that perform particular tasks or implement particular abstract data types. Moreover, those skilled in the art will appreciate that various aspects can be practiced with other computer system configurations, including single-processor or multiprocessor computer systems, mini-computing devices, mainframe computers, as well as computers, hand-held computing devices (e.g., PDA, phone), microprocessor-based or programmable consumer or industrial electronics, and the like. The illustrated aspects can also be practiced in distributed computing environments in which tasks are performed by remote processing devices that are linked through a communications network. However, some, if not all aspects of this disclosure can be practiced on stand-alone computers. In a distributed computing environment, program modules can be located in both local and remote memory storage devices.

As used in this application, the terms “component,” “system,” “platform,” “interface,” and the like, can refer to or can include a computer-related entity or an entity related to an operational machine with one or more specific functionalities. The entities disclosed herein can be either hardware, a combination of hardware and software, software, or software in execution. For example, a component can be, but is not limited to being, a process running on a processor, a processor, an object, an executable, a thread of execution, a program, or a computer. By way of illustration, both an application running on a server and the server can be a component. One or more components can reside within a process or thread of execution and a component can be localized on one computer or distributed between two or more computers. In another example, respective components can execute from various computer readable media having various data structures stored thereon. The components can communicate via local or remote processes such as in accordance with a signal having one or more data packets (e.g., data from one component interacting with another component in a local system, distributed system, or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As yet another example, a component can be an apparatus that provides specific functionality through electronic components without mechanical parts, wherein the electronic components can include a processor or other means to execute software or firmware that confers at least in part the functionality of the electronic components. In an aspect, a component can emulate an electronic component via a virtual machine, e.g., within a cloud computing system.

In addition, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. As used herein, the term “and/or” is intended to have the same meaning as “or.”

Moreover, articles “a” and “an” as used in the subject specification and annexed drawings should generally be construed to mean “one or more” unless specified otherwise or clear from context to be directed to a singular form. As used herein, the terms “example” or “exemplary” are utilized to mean serving as an example, instance, or illustration. For the avoidance of doubt, the subject matter disclosed herein is not limited by such examples. In addition, any aspect or design described herein as an “example” or “exemplary” is not necessarily to be construed as preferred or advantageous over other aspects or designs, nor is it meant to preclude equivalent exemplary structures and techniques known to those of ordinary skill in the art.

The herein disclosure describes non-limiting examples. For ease of description or explanation, various portions of the herein disclosure utilize the term “each,” “every,” or “all” when discussing various examples. Such usages of the term “each,” “every,” or “all” are non-limiting. In other words, when the herein disclosure provides a description that is applied to “each,” “every,” or “all” of some particular object or component, it should be understood that this is a non-limiting example, and it should be further understood that, in various other examples, it can be the case that such description applies to fewer than “each,” “every,” or “all” of that particular object or component.

As it is employed in the subject specification, the term “processor” can refer to substantially any computing processing unit or device comprising, but not limited to, single-core processors; single-processors with software multithread execution capability; multi-core processors; multi-core processors with software multithread execution capability; multi-core processors with hardware multithread technology; parallel platforms; and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures such as, but not limited to, molecular and quantum-dot based transistors, switches and gates, in order to optimize space usage or enhance performance of user equipment. A processor can also be implemented as a combination of computing processing units. In this disclosure, terms such as “store,” “storage,” “data store,” data storage,” “database,” and substantially any other information storage component relevant to operation and functionality of a component are utilized to refer to “memory components,” entities embodied in a “memory,” or components comprising a memory. It is to be appreciated that memory or memory components described herein can be either volatile memory or nonvolatile memory, or can include both volatile and nonvolatile memory. By way of illustration, and not limitation, nonvolatile memory can include read only memory (ROM), programmable ROM (PROM), electrically programmable ROM (EPROM), electrically erasable ROM (EEPROM), flash memory, or nonvolatile random access memory (RAM) (e.g., ferroelectric RAM (FeRAM). Volatile memory can include RAM, which can act as external cache memory, for example. By way of illustration and not limitation, RAM is available in many forms such as synchronous RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), direct Rambus RAM (DRRAM), direct Rambus dynamic RAM (DRDRAM), and Rambus dynamic RAM (RDRAM). Additionally, the disclosed memory components of systems or computer-implemented methods herein are intended to include, without being limited to including, these and any other suitable types of memory.

What has been described above include mere examples of systems and computer-implemented methods. It is, of course, not possible to describe every conceivable combination of components or computer-implemented methods for purposes of describing this disclosure, but many further combinations and permutations of this disclosure are possible. Furthermore, to the extent that the terms “includes,” “has,” “possesses,” and the like are used in the detailed description, claims, appendices and drawings such terms are intended to be inclusive in a manner similar to the term “comprising” as “comprising” is interpreted when employed as a transitional word in a claim.

The descriptions of the various embodiments have been presented for purposes of illustration, but are not intended to be exhaustive or limited to the embodiments disclosed. Many modifications and variations will be apparent without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A system, comprising:
a processor that executes computer-executable components stored in a non-transitory computer-readable memory, wherein the computer-executable components comprise:
an access component that accesses cardiac data associated with a medical patient;
a retrieval component that searches a dynamic cardiology publication repository for one or more cardiology publications that are relevant to the cardiac data of the medical patient and that are published within a threshold margin of a current time or date; and
a generative component that synthesizes a natural language cardiology report for the medical patient, by executing a deep learning neural network on both the cardiac data and the one or more cardiology publications.

2. The system of claim 1, wherein the dynamic cardiology publication repository is a medical website, and wherein the one or more cardiology publications are cardiology research papers or cardiology journal articles published by the medical website.

3. The system of claim 1, wherein the cardiac data comprises: one or more computed tomography images of a heart of the medical patient; one or more magnetic resonance images of the heart of the medical patient; one or more echocardiogram images of the heart of the medical patient; or one or more positron emission tomography images of the heart of the medical patient.

4. The system of claim 1, wherein the cardiac data comprises: an electrocardiogram of the medical patient; a photoplethysmogram of the medical patient; a seismocardiogram of the medical patient; or a phonocardiogram of the medical patient.

5. The system of claim 1, wherein the cardiac data comprises physical characteristics or attributes of the medical patient.

6. The system of claim 1, wherein the retrieval component searches the dynamic cardiology publication repository via: a keyword search technique; an embedding search technique; or a probabilistic information retrieval search technique.

7. The system of claim 1, wherein the computer-executable components further comprise:
a result component that visually renders the natural language cardiology report on an electronic display.

8. The system of claim 7, wherein the result component prompts a user to accept or reject the natural language cardiology report.

9. A computer-implemented method, comprising:
accessing, by a device operatively coupled to a processor, cardiac data associated with a medical patient;
searching, by the device, a dynamic cardiology publication repository for one or more cardiology publications that are relevant to the cardiac data of the medical patient and that are published within a threshold margin of a current time or date; and
synthesizing, by the device and by executing a deep learning neural network on both the cardiac data and the one or more cardiology publications, a natural language cardiology report for the medical patient.

10. The computer-implemented method of claim 9, wherein the dynamic cardiology publication repository is a medical website, and wherein the one or more cardiology publications are cardiology research papers or cardiology journal articles published by the medical website.

11. The computer-implemented method of claim 9, wherein the cardiac data comprises: one or more computed tomography images of a heart of the medical patient; one or more magnetic resonance images of the heart of the medical patient; one or more echocardiogram images of the heart of the medical patient; or one or more positron emission tomography images of the heart of the medical patient.

12. The computer-implemented method of claim 9, wherein the cardiac data comprises: an electrocardiogram of the medical patient; a photoplethysmogram of the medical patient; a seismocardiogram of the medical patient; or a phonocardiogram of the medical patient.

13. The computer-implemented method of claim 9, wherein the cardiac data comprises physical characteristics or attributes of the medical patient.

14. The computer-implemented method of claim 9, wherein the device searches the dynamic cardiology publication repository via: a keyword search technique; an embedding search technique; or a probabilistic information retrieval search technique.

15. The computer-implemented method of claim 9, further comprising:
visually rendering, by the device, the natural language cardiology report on an electronic display.

16. The computer-implemented method of claim 15, further comprising:
prompting, by the device and on the electronic display, a user to accept or reject the natural language cardiology report.

17. A computer program product for facilitating natural language medical reporting via retrieval-augmented generative artificial intelligence, the computer program product comprising a non-transitory computer-readable memory having program instructions embodied therewith, the program instructions executable by a processor to cause the processor to:
access medical data associated with a medical patient;
search a dynamic medical publication repository for one or more medical publications that are relevant to the medical data of the medical patient and that are published within a threshold margin of a current time or date; and
synthesize a natural language medical report for the medical patient, by executing a deep learning neural network on both the medical data and the one or more medical publications.

18. The computer program product of claim 17, wherein the dynamic medical publication repository is a medical website, and wherein the one or more medical publications are medical research papers or medical journal articles published by the medical website.

19. The computer program product of claim 17, wherein the medical data comprises: a body temperature of the medical patient; a pulse rate of the medical patient; a respiration rate of the medical patient; or a blood pressure of the medical patient.

20. The computer program product of claim 17, wherein the medical data comprises a medical scanned image of the medical patient.
